(19) United States
(12) Patent Application Publication
(10) Pub. No.: US 2023/0140260 A1
(43) Pub. Date: May 4, 2023

(54) GREEN METHODS OF MAKING PRODUCT FROM HYDROGEN ENRICHED SYNTHESIS GAS

(71) Applicant: Synata Bio, Inc., Warrenville, IL (US)

(72) Inventor: Jianxin Du, Naperville, IL (US)

(73) Assignee: Synata Bio, Inc., Warrenville, IL (US)

(21) Appl. No.: 18/050,910

(22) Filed: Oct. 28, 2022

Related U.S. Application Data
(60) Provisional application No. 63/273,594, filed on Oct. 29, 2021.

(51) Int. Cl. C12P 7/06; C01B 3/48; C07C 7/148; C25B 1/042; C07C 31/08; C12P 7/54

(52) U.S. Cl. CPC C12P 7/065; C01B 3/48; C07C 7/14866; C25B 1/042; C07C 31/08; C12P 7/54

(57) ABSTRACT

“Green” methods of preparing oxygenated products, animal feed, and fertilizer are disclosed. Desired oxygenated products include, but are not limited to, ethanol, acetic acid, butyrate, butanol, propionate, propanol, or any combination thereof. The methods use synthesis gas (syngas), which can be produced from processing of coal, natural gas, and/or biomass. The syngas contains some combination of hydrogen, carbon monoxide, and/or carbon dioxide. The method entails blending the syngas with purge (tail) gases from industrial processes and/or with hydrogen gas, e.g., produced from renewable sources. The resulting mixture is a H₂-enriched syngas that is fermented by microorganisms that are well suited to ferment hydrogen-rich gases. Byproducts from the method can also be recovered. The disclosure also provides methods of preparing material fertilizer and animal feed, respectively. By repurposing purge gases so they are not emitted into the environment and/or using hydrogen from renewable sources, the disclosed methods are environmentally-friendly.

### CROSS-REFERENCE TO RELATED APPLICATIONS

This patent application claims the benefit of U.S. Provisional Patent Application 63/273,594, filed Oct. 29, 2021, which is incorporated by reference.

### BACKGROUND

It is desirable to use microorganisms to convert certain carbohydrates, such as glucose and sucrose, into a variety of products, such as fuels and chemicals using fermentation. An alternative to production of ethanol by fermentation of carbohydrates is synthesis gas (syngas) fermentation. Syngas is typically derived from the gasification of carbonaceous materials, reforming of natural gas and/or biogas from anaerobic bioreactors (fermentors), or from various industrial methods. The gas substrate generally comprises carbon monoxide, hydrogen, and carbon dioxide and usually contains other components such as water vapor, nitrogen, methane, light hydrocarbons, ammonia, and hydrogen sulfide.

Syngas fermentation is a microbial process, wherein the primary carbon and energy sources are provided from syngas. Commonly referred to as acetogens, these microorganisms utilize small chemical building blocks, present in syngas, in the reductive Acetyl-CoA pathway (Wood-Ljungdahl pathway), to produce ethanol and/or acetic acid. Fermentation of syngas predominantly results in the formation of ethanol and acetic acid. This process requires significant amounts of hydrogen and/or carbon monoxide. The balanced chemical equations for the overall conversion of carbon monoxide, carbon dioxide, and hydrogen to ethanol and acetic acid are as follows:

### Ethanol Production

6CO+3H₂O→C₂H₅OH+4CO₂

6H₂+2CO₂→C₂H₅OH+3H₂O

### Acetic Acid Production

4CO+2H₂O→CH₃COOH+2CO₂

4H₂+2CO₂→CH₃COOH+2H₂O

As demonstrated by the balanced chemical equations, both carbon monoxide and carbon dioxide can be used as the primary source of carbon, facilitated by the electrons present in carbon monoxide and hydrogen.

Climate change is an issue of ever-increasing concern. Greenhouse gases emitted by the manufacturing sector contribute to an increase in the average temperature at the surface of the Earth. Because of increasing concerns regarding climate change, there is a need for additional methods for producing chemicals and fuels that reduce our carbon footprint.

It will be appreciated that this background description has been created by the inventors to aid the reader, and is not to be taken as a reference to prior art nor as an indication that any of the indicated problems were themselves appreciated in the art. While the described principles can, in some regards and embodiments, alleviate the problems inherent in other systems, it will be appreciated that the scope of the protected innovation is defined by the attached claims, and not by the ability of any embodiments of the disclosure to solve any specific problem noted herein.

### BRIEF SUMMARY

The disclosure provides methods of preparing oxygenated products, such as ethanol, acetic acid, butyrate, butanol, propionate, propanol, or any combination thereof, using fermentation by microorganisms. The disclosure also provides methods of preparing material for land use applications such as fertilizer, as well as methods of preparing animal feed. The methods use a synthesis gas (syngas) containing some combination of hydrogen (H₂), carbon monoxide (CO), and/or carbon dioxide (CO₂). The syngas can be produced from a variety of sources including processing of coal, natural gas, petroleum-derivatives, municipal solid waste (hereinafter “MSW”), and/or biomass. The coal-derived H₂-enriched syngas can be in the form of “on purpose” synthesis gas, generally meaning that it is produced as a feedstock for the production of down-stream products. In contradistinction, “purge gas” refers generally to waste gas that is produced as a byproduct from a unit operation. Though purge gas can be used for its fuel value (by combustion to produce heat), it is generally not economical to further process purge gas via separation processes.

Surprisingly and unexpectedly, the syngas can be enriched with hydrogen (H₂) gas to form a H₂-enriched syngas. In some embodiments, industrial purge gases that otherwise would create greenhouse emissions are repurposed in order to enrich the syngas to produce the hydrogen enriched syngas.

Advantageously, the methods of the disclosure can be used as “green” technology. In this regard, hydrogen rich purge gas (sometimes referred to as “tail gas” because it is a waste stream on the tail end of a process) from various industrial processes can be blended with syngas derived from any source (e.g., coal) in order to prepare the H₂-enriched syngas. Hydrogen-rich purge gas refers to a gas that will allow for a higher proportion (relative to other gases) of hydrogen gas in the H₂-enriched syngas upon mixing as compared with the syngas alone. The mixture of the syngas and the hydrogen rich industrial purge (tail) gas is referred to herein as the H₂-enriched syngas (or substrate gas), which can be fermented as described herein. Examples of industrial purge (tail) gas include, but are not limited to, for example, purge gases that are discharged in the production processes of ammonia synthesis, methanol synthesis, acetic acid, ethylene oxidation to ethylene oxide, etc. These industrial tail gases can be produced where coal is available as a feedstock. These processes can be co-located with the coal processing plant to facilitate blending of the coal-derived syngas and the industrial tail gas. Co-location thus means that the syngas production and industrial tail gas production are situated within pipeline distances so that they can be transferred via flow-through pipes.

In some embodiments, hydrogen gas produced by environmentally-friendly, renewable sources such as wind, solar, or a combination thereof, can be used to enrich the syngas with hydrogen gas. For example, the renewable source (e.g., the sun or wind) can be used to generate electricity to run electrolysis to produce hydrogen from water. The use of renewable electricity can be considered a “green” technology in that all compounds can be sourced from renewable sources.

The H₂-enriched syngas is delivered in any suitable manner (e.g., via a compressor or blower) into a bioreactor containing fermentation fluid and a microorganism to form a fermentation broth. The H₂-enriched gas can be desirably fermented using the microorganism, which is selected to be well suited for efficient fermenting of H₂-enriched syngas to produce an oxygenated product in the broth. For example, the microorganism can be in the form of acetogenic carboxydotrophic bacteria, such as, for example, Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

The oxygenated product can be separated from the broth by any suitable means as will be understood in the art. For example, the oxygenated product can be separated by fractional distillation, evaporation, pervaporation, gas stripping, phase separation, extractive fermentation, including for example, liquid-liquid extraction, or any combination thereof. The bacteria are removed from the broth by any suitable solid/liquid separation technology such as centrifugation or filtration. The remaining constituents of the broth can be treated by liquid/liquid or liquid/vapor separation processes such as distillation in order to purify product streams. The remaining solids are consolidated and can be used for fertilizer and/or animal feed, e.g., depending on market conditions and regulatory approval.

As a result, the methods of the disclosure are “green” and environmentally friendly. In some embodiments, industrial tail gases are repurposed with regard to pollution control. Instead of burning the industrial tail gases for release into the atmosphere, tail gas is captured and repurposed by accumulating it in the syngas (to increase the relative hydrogen gas content therein) used in producing oxygenated product, animal feed, and/or fertilizer. The hydrogen in the tail gas can be derived from e.g., methanol or ammonia. In some embodiments, the hydrogen content is increased in the syngas by inserting hydrogen from environmentally-friendly sources such as wind and/or solar. Furthermore, when the oxygenated product is ethanol, there are additional environmental benefits inasmuch as ethanol is considered a green fuel because it is nontoxic and reduces air pollution. In this regard, the use of ethanol in fuel has been found to reduce greenhouse gas emissions.

Thus, in one aspect, the disclosure provides a method of preparing an oxygenated product, in which the method uses acetogenic carboxydotrophic bacteria. The method comprises providing a syngas comprising at least two of the following components: CO, CO₂, and H₂. Particularly, the syngas is enriched with hydrogen gas, e.g., by blending the syngas with a H₂ rich gas (e.g., industrial tail gas and/or renewably produced hydrogen gas) to form the H₂-enriched syngas. The H₂-enriched syngas is fermented with acetogenic carboxydotrophic bacteria (e.g., in a liquid medium to form a broth in a bioreactor) to produce an oxygenated product in the broth. The oxygenated product can be separated from the broth by known techniques such as those discussed herein.

In another aspect, the disclosure provides a method of preparing an oxygenated product in which the H₂ content in the syngas is enriched to at least about 50 vol. % of H₂. The method comprises providing a syngas comprising at least two of the following components: CO, CO₂, and H₂. The H₂ content from the syngas is enriched to form the H₂-enriched syngas having at least about 50 vol. % of H₂, e.g., from about 50 vol. % to about 85 vol. %, from about 50 vol. % to about 70 vol. %, or from about 60 vol. % to about 70 vol. % of H₂. Particularly, the syngas is enriched with hydrogen gas, e.g., by blending the syngas with a H₂ rich gas (e.g., industrial tail gas and/or renewably produced hydrogen gas) to form the H₂-enriched syngas. The H₂-enriched syngas is fermented with bacteria (e.g., in a liquid medium to form a broth in a bioreactor) to produce an oxygenated product in the broth. The oxygenated product can be separated from the broth by known techniques such as those discussed herein.

In another aspect, the disclosure provides a method of preparing an oxygenated product in which the H₂-enriched syngas has an e/C of at least about 5.7. As referred to herein, the e/C is a calculated ratio of the total number of electrons available for reaction as provided from syngas components, namely H₂ and CO, divided by the total moles of C-carbon in syngas. H₂ and CO each contain two electrons per molecule that are available for chemical reactions. CO₂ is included in the carbon balance but provides no electrons for chemical reactions. While CH₄ also contains ‘C’ and electrons, it is considered an inert compound in syngas fermentation and is therefore not included in e/C calculations. The e/C indicates hydrogen content in the gas mixture because hydrogen contributes electrons but carbon does not. The method comprises providing a syngas comprising at least two of the following components: CO, CO₂, and H₂. The H₂ content in the substrate gas is enriched so that the H₂-enriched syngas has an e/C of at least about 5.7, e.g., from about 5.7 to about 8.0. Particularly, the syngas is enriched with hydrogen gas, e.g., by blending the syngas with a H₂ rich gas (e.g., industrial tail gas and/or renewably produced hydrogen gas) to form the H₂-enriched syngas. The H₂-enriched syngas is fermented with bacteria (in a liquid medium to form a broth in a bioreactor) to produce an oxygenated product in the broth. The oxygenated product can be separated from the broth by known techniques such as those discussed herein.

In another aspect, the disclosure provides a method of renewably preparing an oxygenated product. The method comprises providing a syngas comprising at least two of the following compounds: CO, CO₂, and H₂. H₂ from a renewable source is blended with the syngas to form an H₂-enriched syngas. The renewable source for the H₂ generates electricity to run electrolysis to produce renewable hydrogen. The renewable source for the H₂ can be, for example, solar, wind, or a combination thereof. The H₂-enriched syngas is fermented with bacteria such as acetogenic carboxydotrophic bacteria (e.g., in a liquid medium to form a broth in a bioreactor) to produce an oxygenated product in the broth. The oxygenated product can be separated from the broth by known techniques such as those discussed herein.

In another aspect, the disclosure provides a method of preparing an animal feed. As used herein, animal feed can be of any suitable type, such as, for example, aquatic culture (fish feed), poultry feed, cattle feed, hog feed, bird feed, etc. The method comprises providing a syngas comprising at least two of the following components: CO, CO₂, and H₂. The H₂ content in the H₂-enriched syngas is enriched to form H₂-enriched syngas having, e.g., (i) at least about 50 vol. % of H₂, such as from about 50 vol. % to about 85 vol. %, from about 50 vol. % to about 70 vol. %, or from about 60 vol. % to about 70 vol. % of H₂, and/or (ii) an e/C of at least about 5.7, such as from about 5.7 to about 8.0. Particularly, the syngas is enriched with hydrogen gas, e.g., by blending the syngas with a H₂ rich gas (e.g., industrial tail gas and/or renewably produced hydrogen gas) to form the H₂-enriched syngas. The H₂-enriched syngas is fermented with bacteria, such as acetogenic carboxydotrophic bacteria (e.g., in a liquid medium to form a broth in a bioreactor) to produce an oxygenated product and a solid byproduct in the broth. The oxygenated product is separated from the broth to produce an oxygenated product-depleted broth. The oxygenated product can be separated from the broth by known techniques such as those discussed herein. The solid byproduct from the broth and/or the oxygenated product-depleted broth is removed (e.g., by centrifugation or filtration) to produce a concentrated biosolid fraction and a clarified stream filtrate, the concentrated biosolids being effective for use as animal feed. The clarified stream filtrate can optionally be treated as wastewater or recycled back to the process, if desired.

In another aspect, the disclosure provides a method of preparing fertilizer. The method comprises providing a syngas comprising at least two of the following components: CO, CO₂, and H₂. The syngas is enriched with H₂ to form H₂-enriched syngas having, e.g., (i) at least about 50 vol. % of H₂, such as from about 50 vol. % to about 85 vol. %, from about 50 vol. % to about 70 vol. % or from about 60 vol. % to about 70 vol. % of H₂, and/or (ii) an e/C of at least about 5.7, such as from about 5.7 to about 8.0. Particularly, the syngas is enriched with hydrogen gas, e.g., by blending the syngas with a H₂ rich gas (e.g., industrial tail gas and/or renewably produced hydrogen gas) to form the H₂-enriched syngas. The H₂-enriched syngas is fermented with bacteria, such as acetogenic carboxydotrophic bacteria (e.g., in a liquid medium to form a broth in a bioreactor) to produce an oxygenated product and a solid byproduct in the broth. The oxygenated product is separated from the broth to produce an oxygenated product-depleted broth. The oxygenated product can be separated from the broth by known techniques such as those discussed herein. The solid byproduct from the broth and/or the oxygenated product-depleted broth is removed (e.g., by centrifugation or filtration) to produce a concentrated biosolid fraction and a clarified stream filtrate, the concentrated biosolids being effective for use as a fertilizer. The clarified stream filtrate can optionally be treated as wastewater or recycled back to the process, if desired.

It will be understood that the preceding aspects are not limited by the descriptions above. Sub-aspects are described in the Detailed Description below, taken with the figures and examples, etc. It will be further understood that various sub-aspects including components, ingredient types, amounts, and properties, as well as other parameters, ranges, and other details described herein are fully contemplated in connection with the aspects above and they can be incorporated as desired into the aspects of the preceding paragraphs unless directly contradicted or expressly excluded.

### BRIEF DESCRIPTION OF THE SEVERAL VIEWS OF THE DRAWING(S)

FIG. 1 is a flow chart depicting the processing of syngas production and cleanup in accordance with embodiments of the present disclosure.

FIG. 2 is a flow chart depicting the process of acetic acid production using methanol in accordance with embodiments of the present disclosure.

FIG. 3 is a flow chart depicting the process of ethylene glycol production through coal gasification in accordance with embodiments of the present disclosure.

FIG. 4 is a flow chart depicting the process of ethanol production by microbial fermentation by mixing hydrogen rich industrial tail gas with coal-derived syngas in accordance with embodiments of the present disclosure.

FIG. 5 is a flow chart depicting the process of ethanol production by microbial fermentation by reforming hydrogen rich industrial tail gas with waste carbon dioxide containing streams in accordance with embodiments of the present disclosure.

FIG. 6 is a flow chart depicting the process of ethanol production by microbial fermentation by direct feed to fermentation of carbon monoxide rich industrial tail gas in accordance with embodiments of the present disclosure.

FIG. 7 is a flow chart depicting the process of ethanol production by microbial fermentation by reforming carbon monoxide rich industrial tail gas by water gas shift in accordance with embodiments of the present disclosure.

FIG. 8 is a flow chart depicting the process of ethanol production by microbial fermentation by mixing reforming carbon monoxide rich industrial tail gas with renewable hydrogen (carbon fixing with renewable hydrogen) in accordance with embodiments of the present disclosure.

### DETAILED DESCRIPTION

Embodiments of the disclosure provide “green” methods of preparing oxygenated product, land application material such as fertilizer, and/or animal feed. In some embodiments, carbon emissions can be reduced by repurposing certain factory waste emissions so that they are used in production of desired products such as biofuels, chemicals, animal feed, and fertilizer, instead of being discharged into the natural environment.

In some embodiments, hydrogen gas from “green,” renewable sources such as solar and wind are used in the production of the fuels, chemicals, animal feed, and fertilizer. In some embodiments, the animal feed can be in the form of fish feed, poultry feed, cattle feed, hog feed, bird feed, etc. Surprisingly and unexpectedly, the present inventors have found that the use of “green” sources of electricity in electrolysis to form hydrogen from water advantageously avoids the need for a water gas shift reaction (conventionally used to enrich the hydrogen content in coal-based syngas) which generates CO₂ as a pollutant. Advantageously, by avoiding the use of the water gas shift reaction and using microbial fermentation, the need for additional steps to ensure the removal of, among other things, H₂S and CO₂ from the syngas is thereby rendered unnecessary. Surprisingly and unexpectedly, in accordance with embodiments of the disclosure, the inventors have found that the presence of H₂S enhances the efficiency of the process as it can be used to offset the need for supplemental sources of sulfur. The inventors have also found that the process is not necessarily undesirably impacted by the presence of CO₂, further rendering the need for “cleanup” steps unnecessary.

### Methods of Preparing Oxygenated Product, Animal Feed and Fertilizer

Synthesis gas (syngas) having a particular composition derived from coal can be used as a starting material. In this regard, generally, as coal is oxidized during the gasification process, it produces syngas. Syngas contains carbon monoxide, hydrogen, and/or carbon dioxide in some proportion, depending on, e.g., the type of gasification process. The inventors have discovered that, surprisingly and unexpectedly, the syngas can be mixed with industrial purge gases (waste gas) to raise the proportion of hydrogen gas content and/or to achieve a particular higher e/C (indicating higher hydrogen content in the ratio of CO/H₂:CO₂) in the resulting H₂-enriched syngas to be fermented. The purge gases are selected so that they increase the hydrogen content or e/C in the H₂-enriched syngas. By way of example, but not limitation, the purge gas can be derived from production of methanol, ammonia, and/or coke oven gas. In some embodiments, purge gases from the production of acetic acid, ethylene glycol, steel mill gas, and/or calcium carbide furnace tail gas can be added to syngas to control the hydrogen content. In some embodiments, the syngas is mixed with hydrogen gas, e.g., obtained by electrolysis using renewable sources such as a wind, solar, or a combination thereof in order to achieve the desired hydrogen gas content and/or e/C.

Generally, the H₂-enriched syngas is fed into a bioreactor of any desired size or type containing fermentation fluid and bacteria to form a fermentation broth. In some embodiments, the bioreactor is industrial sized, having a capacity of, for example, tens of thousands, hundreds of thousands, or even a million liters or more. The bioreactor can be of any suitable type of design as will be understood in the art. The bioreactor can be in any suitable form, e.g., a tank with suitable mixing capability. In some embodiments, the bioreactor contains an agitator (e.g., an impeller) to facilitate mixing of the constituents added to the bioreactor. Alternatively, mixing can be achieved without an impeller by the pumping of liquid and/or the injection of gas into the bioreactor. For example, the tank can be cylindrical or other shape and the agitator (e.g., impeller) can be motor driven. For example, for gas fermentation, the bioreactor can be in the form of a continuously stirred tank reactor (CSTR), bubble column, air lift reactor, etc.

Ingredients including at least water, H₂-enriched syngas, microorganism, nutrients, and vitamins are added to the bioreactor to form a fermentation broth therein to allow for the fermentation process. Each component can be delivered to the bioreactor in any suitable manner, e.g., via a recycled or new stream with the aid of a pump, gas nozzle, solid metering or other desired techniques. The water is useful as a transfer agent by delivering nutrients and other components. It is also well suited as a medium in the bioreactor as it can be readily stirred and allows for growth of microbes in a suspension while also accommodating subsequent separation of various components.

In some embodiments, fermentation fluid contains from about 95% to about 99% water, vitamins in an amount of about 0.01% or less, nutrients in an amount of about 1% to about 2.5% (where all amounts are by weight of the component per 100 ml, as appreciated by one of ordinary skill in the art). Vitamins and nutrients useful for inclusion in the fermentation fluid are known (see, e.g., U.S. Pat. No. 6,340,581 B1, which description of vitamins and nutrients is incorporated by reference herein).

During fermentation, the bacteria functions to convert the H₂, CO, and CO₂ present in the H₂-enriched syngas in accordance with the Wood-Ljungdahl pathway in order to form an oxygenated product, as well as biosolids as a byproduct. In this regard, carbon is provided by CO and/or CO₂. Energy is provided by CO and/or H₂.

The bacteria and the oxygenated product are each separated from the fermentation broth. The bacteria can be separated by centrifugation or filtration. In some embodiments, the oxygenated product is separated by fractional distillation, evaporation, pervaporation, gas stripping, phase separation, and extractive fermentation, including for example, liquid-liquid extraction, or any combination thereof. After removal of the biosolids and oxygenated product the resulting clarified stream can be returned to the reactor, or treated by aerobic or anaerobic digestion.

Instead of adding to greenhouse emissions and raising carbon footprint, the purge gases are mixed into the H₂-enriched syngas and fermented as described herein to produce chemicals and fuels. As such, embodiments of the disclosure provide significant green technology via carbon capture and reduction in greenhouse gases and hence carbon footprint.

Methods of the disclosure include, e.g., a method of preparing an oxygenated product, a method of preparing animal feed, and a method of making fertilizer. The method comprises providing a syngas comprising at least two of the following components: CO, CO₂, and H₂. The syngas is enriched with hydrogen (by blending the syngas with an industrial tail gas or hydrogen gas from renewable sources, as described herein) so that (a) the H₂ content in the H₂-enriched syngas is at least about 50 vol. % of H₂, e.g., from about 50 vol. % to about 70 vol. %, or from about 60 vol. % to about 70 vol. % of H₂; and/or (b) the H₂-enriched syngas has an e/C of at least about 5.7, e.g., from about 5.7 to about 8.0. The H₂-enriched syngas is fermented by microorganisms suited to ferment H₂-enriched syngas (e.g., acetogenic carboxydotrophic bacteria) in a liquid medium forming a broth in a bioreactor to produce an oxygenated product in the broth. The oxygenated product can be recovered from the broth by known techniques, e.g., as described herein.

In some embodiments, the H₂-enriched syngas has an e/C of at least about 5.7, e.g., from about 5.7 to about 8.0. The H₂-enriched syngas can have any suitable e/C, e.g., an e/C from about 5.7 to 6.0, or from 5.7 to 6.1, or from 5.7 to 6.2, or from 5.7 to 6.3, or from 5.7 to 6.4, or from 5.7 to 6.5, or from 5.7 to 6.6, or form 5.7 to 6.7, or from 5.7 to 6.8, or from 5.7 to 6.9, or from 5.7 to 7.0, or from 5.7 to 7.1, or from 5.7 to 7.2, or from 5.7 to 7.3, or from 5.7 to 7.4, or from 5.7 to 7.5, or from 5.7 to 7.6, or from 5.7 to 7.7, or from 5.7 to 7.8, or from 5.7 to 7.9, or from 5.7 to 8.

In some embodiments, the method for preparing an oxygenated product uses renewable H₂. In this regard, H₂ gas is added from renewable sources (instead of, or in addition to, from industrial purge gases) into the syngas to form H₂-enriched syngas. The H₂ gas can be provided by suitable renewable sources such as solar, wind, or a combination thereof. The renewable source for the H₂ generates electricity to run electrolysis to produce renewable hydrogen. Thus, the method comprises providing a syngas comprising at least two of the following compounds: CO, CO₂, and H₂; adding H₂ from a renewable source to the H₂-enriched syngas to form an H₂-enriched syngas; fermenting the H₂-enriched syngas with microorganisms (e.g., acetogenic carboxydotrophic bacteria) in a liquid medium to form a broth in a bioreactor to produce oxygenated product in the broth. The oxygenated product can be recovered from the broth by known techniques, e.g., as described herein.

In accordance with some embodiments, byproducts of the process for making the oxygenated compound can be captured and used for applications such as fertilizer and/or animal feed. In this respect, after the H₂-enriched syngas is fermented by the microorganism (e.g., acetogenic carboxydotrophic bacteria), an oxygenated product and a solid byproduct containing biosolids are produced in the broth. The oxygenated product can be recovered from the broth so it can be prepared for its intended use. The solid byproduct can be removed before or after the removal of the oxygenated product, e.g., by way of, e.g., centrifugation and filter press, etc. to produce a cake and a clarified stream filtrate. The clarified stream filtrate can be recycled back into the fermentation fluid for additional fermentation cycles. The cake is a mass of the biosolid particles and can be effective for use as a fertilizer and/or animal feed (optionally, after a drying step). The respective compositions of the animal feed and fertilizer are generally similar because they are mainly composed of microbial proteins and/or carbohydrates. In some embodiments, the animal feed and/or fertilizer contains protein (e.g. from about 30 wt. % to about 90 wt. %, such as from about 60 wt. % to about 90 wt. %), fat (e.g. from about 1 wt. % to about 12 wt. %, such as from about 1 wt. % to about 3 wt. %), carbohydrate (e.g. from about 5 wt. % to about 60 wt. %, such as from about 15 wt. % to about 60 wt. %, or from about 5 wt. % to about 15 wt. %) and/or minerals such as sodium, potassium, copper etc. (e.g. from about 1 wt. % to about 20 wt. %, such as from about 1 wt. % to about 3 wt. %). For example, the animal feed and/or fertilizer can contain about 86% protein, about 2% fat, about 2% minerals, and about 10% carbohydrate.

Thus, in a method of preparing an animal feed, the method comprises: (a) providing a syngas comprising at least two of the following components: CO, CO₂, and H₂; (b) enriching the H₂ content in the syngas (by blending the syngas with, e.g., an industrial tail gas and/or hydrogen gas from renewable sources, as described herein), e.g., (i) to at least about 50 vol. % of H₂, such as from about 50 vol. % to about 85 vol. %, from about 50 vol. % to about 70 vol. % or from about 60 vol. % to about 70 vol. % of H₂, and/or (ii) to an e/C of at least about 5.7, such as from about 5.7 to about 8.0; (c) fermenting the H₂-enriched syngas with bacteria, such as acetogenic carboxydotrophic bacteria, in a liquid medium to form a broth in a bioreactor to produce an oxygenated product and a solid byproduct in the broth; (d) removing the oxygenated product from the broth to produce an oxygenated product-depleted broth; and (e) removing the solid byproduct from the broth and/or the oxygenated product-depleted broth to produce a cake and a clarified stream filtrate, the cake being effective for use as wet or dry animal feed. It will be understood that steps (d) and (e) can be performed in either order. In some embodiments, the method further comprises drying the cake, the dried cake effective as a dry animal feed. In some embodiments, the cake is dried to enhance stability and/or for ease of transport and/or storage, but can optionally be mixed with water prior to use.

The animal feed can be in the form of aquatic culture (fish feed), poultry feed, cattle feed, hog feed, bird feed, etc. In the case of fish feed, in some embodiments, advantageously, the fish feed can avoid high contents of metals such as mercury. In some embodiments, desirably, the fish feed can be prepared without the high contents of metals such as mercury while also having a relatively high content of amino acids.

In a method of preparing fertilizer, the method comprises: (a) providing a syngas comprising at least two of the following components: CO, CO₂, and H₂; (b) enriching the H₂ content in the syngas (by blending the syngas with an industrial tail gas or hydrogen gas from renewable sources, as described herein), e.g., (i) to at least about 50 vol. % of H₂, such as from about 50 vol. % to about 85 vol. %, from about 50 vol. % to about 70 vol. % or from about 60 vol. % to about 70 vol. % of H₂, and/or (ii) to an e/C of at least about 5.7, such as from about 5.7 to about 8.0; (c) fermenting the H₂-enriched syngas with bacteria, such as acetogenic carboxydotrophic bacteria, in a liquid medium to form a broth in a bioreactor to produce an oxygenated product and a solid byproduct in the broth; (d) removing the oxygenated product from the broth to produce an oxygenated product-depleted broth; and (e) removing the solid byproduct from the broth and/or the oxygenated product-depleted broth to produce a cake and a clarified stream filtrate, the cake being effective for use as a wet or dry fertilizer. Steps (d) and (e) can be performed in either order. In some embodiments, the method further comprises drying the cake, the dried cake effective as a dry fertilizer. In some embodiments, the cake is dried to enhance stability and/or for ease of transport and/or storage, but can optionally be mixed with water prior to use.

### Syngas

Syngas can be formed from a variety of sources containing carbon, hydrogen, and oxygen. For example, useful carbon/hydrogen/oxygen materials include natural gas and materials that can be gasified, such as coal, biomass, discarded materials such as MSW. Certain sources, e.g., enriched natural gas, may be liquefied to beneficially transport it across long distances but could also be generated in situ and piped-in on location.

Syngas from any suitable source and containing any suitable ratio of carbon monoxide/hydrogen/carbon dioxide can be used. Generally, however, the syngas will have less hydrogen content than H₂-enriched syngas as described herein. Typically, the source syngas has an e/C of at least about 2, e.g., from about 2 to about 5.7. In this regard, the e/C indicates the ratio of total number of electrons to carbon atoms and the syngas normally will have a lower e/C (as compared with the H₂-enriched syngas). As discussed herein, the syngas is blended with, e.g., industrial tail gas and/or hydrogen from renewable sources such that the resulting H₂-enriched syngas s is characterized by a hydrogen content and/or e/C that are higher than the hydrogen content and/or e/C of the syngas alone.

The syngas can be desirably derived from coal dependent processes. This method for H₂ enrichment is particularly useful because coal derived syngas has a reduced e/C. The precise proportion of CO:H₂:CO₂ in the syngas will vary depending on the starting material and, e.g., if present, the degree of water-gas shift carried out after gasification.

The syngas can generally have any suitable hydrogen content, although the hydrogen content will be less than that of the H₂-enriched syngas (i.e., after the syngas is blended with the industrial tail gas and/or hydrogen gas from renewable sources). For example, in some embodiments, the syngas contains from about 5 vol. % to about 80 vol. % of H₂, or from about 50 vol. % to about 80 vol. % of H₂.

The syngas can generally have any suitable carbon monoxide content. For example, in some embodiments, the syngas contains from about 3 vol. % to about 85 vol. % of CO, e.g., from about 10 vol. % to about 50 vol. % of CO. In some embodiments, the syngas will have a higher relative volume percentage of carbon monoxide as compared with the blended H₂-enriched syngas.

The syngas can generally have any suitable carbon dioxide content. For example, in some embodiments, the syngas contains from about 0 vol. % to about 45 vol. % of CO₂, e.g., from about 3 vol. % to about 45 vol. % of CO₂ or from about 3 vol. % to about 25 vol. % of CO₂. In some embodiments, the syngas will have a higher relative volume percentage of carbon dioxide as compared with the blended H₂-enriched syngas.

### Industrial Purge (Tail) Gases

In some embodiments, the syngas is blended with an industrial purge gas to form the H₂-enriched syngas. Purge gas is generally an exhaust gas that is discharged in the production of many chemicals or materials. Purge gas is sometimes referred to as a tail gas because it is part of the exhaust stream. The use of coal-derived purge gas is particularly useful in embodiments of the disclosure due to its abundance and continuous supply.

In order to maintain, e.g., chemical reaction balance, high efficiency, and normal and stable operation, gas generated by a side reaction in the chemical process or the remaining components of the raw material mixed gas are often discharged out of a production unit continuously or periodically for all or part of the low-grade gas components that can no longer be used in the chemical process. Low grade gas components refer to low content of effective gas components and high content of impurities. The part of gas that is discharged in the process is called purge gas. For example, a large number of purge gases are discharged in the production processes of ammonia synthesis, methanol synthesis, acetic acid, ethylene oxidation to ethylene oxide, etc. Purge gas is different from the gas temporarily discharged due to accidents, abnormal production, equipment cleaning, replacement and other processes.

For example, purge gas can be derived from methanol production. An exemplary composition of purge gas from methanol production is set forth in Table 1. The potential volume of purge gas derived from methanol production is approximately 300 Nm³ per ton-methanol (equivalent to about 0.05 ton-ethanol per ton-methanol). The potential ethanol production volume in China alone of purge gas derived from methanol production is up to 2.5 million tons-ethanol (based on 50 million tons-methanol production in 2019). Current uses of purge gas derived from methanol production include burning in the flare, burning in a waste heat boiler for energy recovery (BTU value), and hydrogen recovery. Representative compositions of purge gas from methanol production, in accordance with some embodiments of the present disclosure, are provided in Table 1.

TABLE 1

| Component | Volume % |
|---|---|
| H₂ | 65-80% |
| CO | 3-5% |
| CO₂ | 5-7% |
| CH₄ | 1-3% |
| N₂ | 5-10% |
| H₂O | 0.5-1% |
| MeOH | 0.5-1% |
| Ar | — |
| Other | — |

As another example, purge gas can also be derived from synthetic ammonia production. The composition of purge gas from synthetic ammonia production is set forth in Table 2. The potential volume of purge gas derived from synthetic ammonia production is approximately 100 Nm³ per ton-ammonia (equivalent to about 0.02 ton-ethanol per ton-ammonia). The potential ethanol production volume in China alone of purge gas derived from synthetic ammonia production is up to 1.5 million tons (based on 70 million tons-ammonia production in 2019). Current uses of purge gas derived from synthetic ammonia production include burning in the flare, burning in a waste heat boiler for energy recovery (BTU value), and hydrogen recovery. Representative compositions of purge gas from synthetic ammonia production, in accordance with some embodiments of the present disclosure, are provided in Table 2.

TABLE 2

| Component | Volume % |
|---|---|
| H₂ | 60-70% |
| CO | — |
| CO₂ | — |
| CH₄ | 5-10% |
| N₂ | 20-25% |
| H₂O | — |
| Ammonia | <200 ppm |
| Ar | 3-8% |
| Other | — |

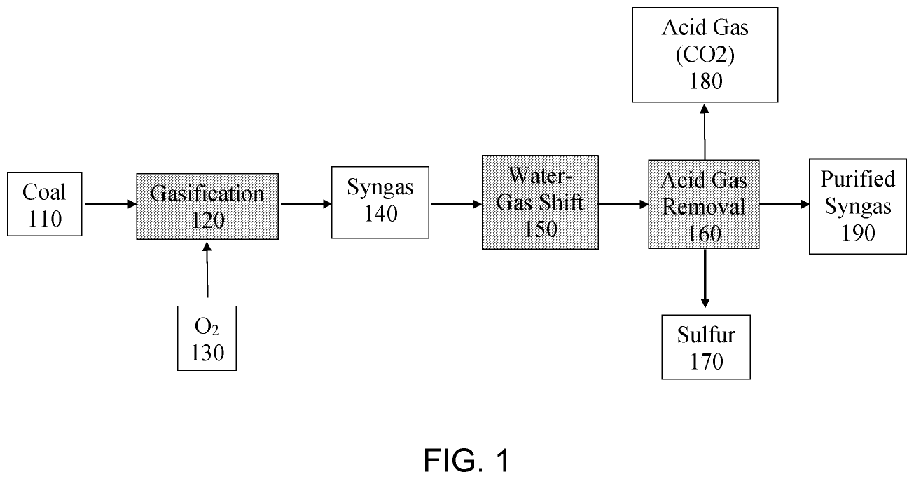

An embodiment for preparing syngas from the gasification of coal is reflected in FIG. 1. As seen in FIG. 1, coal 110 is subjected to gasification 120 with introduction of oxygen 130 to produce a CO-rich syngas 140. This syngas is subjected to water gas shift 150 to increase H₂ content, followed by acid gas removal 160. Acid gas refers to a gas mixture containing hydrogen sulfide (H₂S), carbon dioxide (CO₂), or related acidic gases. Acid gas removal result in three streams: a purified form of syngas suitable for chemical conversion 190, a H₂S rich stream 170, and an acid gas stream enriched in CO₂ 180. The composition of acid gas is listed in Table 3. Current uses for raw acid gas include discharging into atmosphere (as a major greenhouse gas from the coal chemical industry). In addition, purified acid gas is used as CO₂ for beverage, dry ice manufacturing. Representative compositions for acid gas are provided, in accordance with some embodiments of the present disclosure, are provided in Table 3.

TABLE 3

| Component | Volume % |
|---|---|
| H₂ | <0.1% |
| CO | <0.5% |
| CO₂ | 95-99% |
| CH₄ | <0.1% |
| N₂ | <0.5% |
| Ar | <0.1% |
| Other | — |

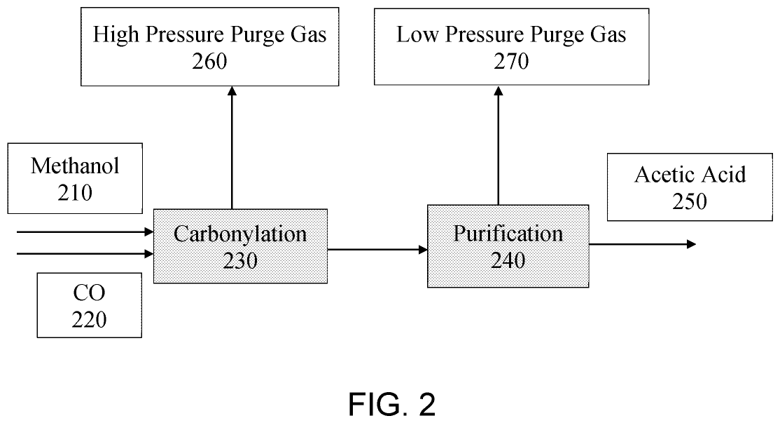

In some embodiments, purge gas can be derived from acetic acid production. The process of acetic acid production using methanol, in accordance with some embodiments, can be seen in FIG. 2. As seen in FIG. 2, methanol 210 and CO 220 undergo carbonylation 230 and purification 240 to produce acetic acid 250. High pressure purge gas 260 is produced during carbonylation 230 and low pressure purge gas 270 is produced during purification 240. Current uses of purge gas derived from acetic acid production include burning in the flare and burning in a waste heat boiler for energy recovery (BTU value). Representative compositions for high pressure purge gas and low pressure purge gas are provided, in accordance with some embodiments of the present disclosure, in Tables 4 and 5, respectively.

TABLE 4

| Component | Vol % |
|---|---|
| H₂ | 1-2% |
| CO | 70-80% |
| CO₂ | 4-5% |
| CH₄ | 6-7% |
| N₂ | 4-10% |
| Ar | — |
| CH₃OH | <0.01% |
| Other | — |

TABLE 5

| Component | Vol % |
|---|---|
| H₂ | 1-2% |
| CO | 60-70% |
| CO₂ | 10-15% |
| CH₄ | 8-10% |
| N₂ | 7-10% |
| Ar | — |
| CH₃OH | <0.01% |
| Other | — |

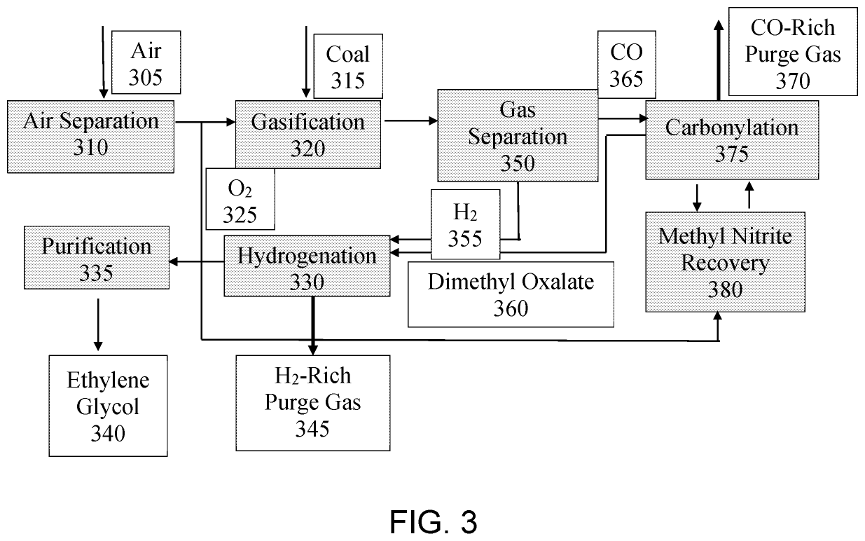

Purge gas can be derived from ethylene glycol production, in accordance with some embodiments. The process of ethylene glycol production through coal gasification, according to some embodiments, can be seen in FIG. 3. Air 305 is subjected to air separation 310 and used in the gasification 320 of coal 315. The gasified material is then subjected to gas separation 350 and mixed with CO 365 to carry out carbonylation 375, which produces a CO-rich purge gas 370 stream. After carbonylation the material is either subjected to methyl nitrite recovery 380 or subjected to hydrogenation 330 using H₂ 355, which produces a H₂ rich purge gas 345. The product is then purified 335 to produce ethylene glycol 340. Representative compositions for CO-rich purge gas and H₂-rich purge gas, in accordance with some embodiments of the present disclosure, are provided in Tables 6 and 7, respectively. Current uses of purge gas derived from ethylene glycol production include burning in the flare and burning in a waste heat boiler for energy recovery (BTU value).

TABLE 6

| Component | Volume % |
|---|---|
| H₂ | 1-2% |
| CO | 65-75% |
| CO₂ | 5-10% |
| CH₄ | 5-10% |
| N₂ | 5-10% |
| Ar | — |
| Other | — |

TABLE 7

| Component | Volume % |
|---|---|
| H₂ | 70-80% |
| CO | 3-5% |
| CO₂ | 5-10% |
| CH₄ | 5-10% |
| N₂ | 5-10% |
| Ar | — |
| Other | — |

In some embodiments, calcium carbide furnace tail gas can be used as the purge gas. Representative compositions for calcium carbide furnace tail gas, in accordance with some embodiments of the present disclosure, are set forth in Table 8. The potential volume of calcium carbide furnace tail gas is approximately 400 Nm³ per ton-calcium carbide (equivalent to about 0.1 ton-ethanol/ton-calcium carbide). The potential ethanol production volume in China alone of calcium carbide furnace tail gas is up to 3.0 million tons (based on 30 million tons-calcium carbide production in 2019). Current uses of calcium carbide furnace tail gas include burning in waste heat boiler for energy recovery (BTU value), coke drying, and power generation.

TABLE 8

| Component | Volume % |
|---|---|
| H₂ | 2-10% |
| CO | 75-85% |
| CO₂ | 2-10% |
| CH₄ | 2-4% |
| N₂ | 1-8% |
| O₂ | <0.5% |
| Other | 1-5% |

In some embodiments, coke oven gas (COG) can be used as the purge gas. Representative compositions for coke oven gas, in accordance with some embodiments of the present disclosure, are set forth in Table 9. The potential volume of coke oven gas is approximately 420 Nm³ per ton-Coke (equivalent to about 0.08 ton-ethanol/ton-calcium carbide. The potential ethanol production volume in China alone of coke oven gas is up to 36 million tons (based on 450 million tons-calcium carbide production in 2019). Current uses of coke oven gas include burning to heat coke oven (BTU value)-40-45% of the total COG, power generation, and ammonia/methanol/NG synthesis.

TABLE 9

| Component | Volume % |
|---|---|
| H₂ | 55-60% |
| CO | 5-8% |
| CO₂ | 1.5-3% |
| CH₄ | 25-28% |
| N₂ | 3-7% |
| O₂ | <0.5% |
| C₂H₂ | 2-4% |

In some embodiments, steel mill gas (SMG) can be used, e.g., to lower the e/C. Representative compositions for steel mill gas, in accordance with some embodiments of the present disclosure, are set forth in Table 10. For example, steel mill gas can be produced from a blast furnace during steel production. It contains CO and CO₂ with small amount of H₂. In some embodiments, the SMG can be used as an additional (third) input gas along with synthesis gas and a hydrogen rich gas to achieve a specific e/C.

TABLE 10

| Component | Volume % |
|---|---|
| H₂ | 2-5% |
| CO | 20-25% |
| CO₂ | 20-25% |
| CH₄ | 2-5% |
| N₂ | 40-50% |
| Ar | — |

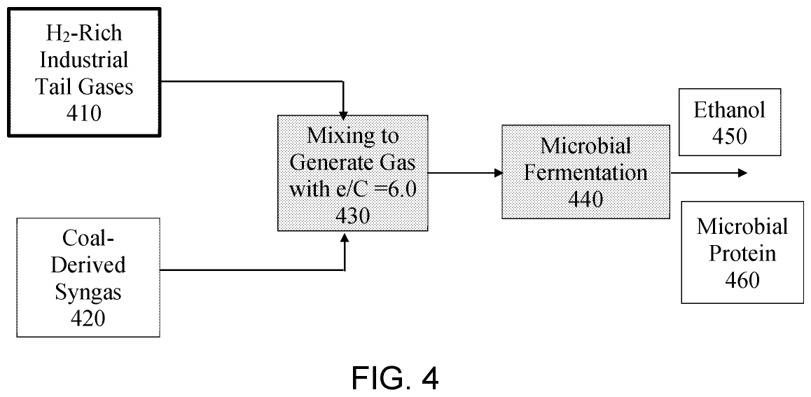

In accordance with embodiments of the disclosure, industrial tail gas can be used for ethanol production by microbial fermentation. Oxygenated product (e.g., ethanol) can be produced by microbial fermentation using H₂-rich industrial tail gases, such as methanol purge as, ammonia purge gas, coke oven gas (COG) etc. Embodiments of the process of producing ethanol by microbial fermentation by mixing hydrogen rich industrial tail gas with coal-derived syngas is set forth in FIG. 4. As seen in FIG. 4, H₂-rich industrial tail gases 410 are mixed with coal-derived syngas 420 to generate gas 430 with e/C of, e.g., at least about 5.7 (such as from about 5.7 to about 8.0). The hydrogen-enriched syngas 430 is then used as source of carbon and energy for microbial fermentation 440 resulting in the production of ethanol 450 and microbial protein 460. Broth is removed from the reactor and ethanol 450 is recovered by techniques such as distillation. Biosolids enriched in microbial protein 460 are also recovered from the removed broth.

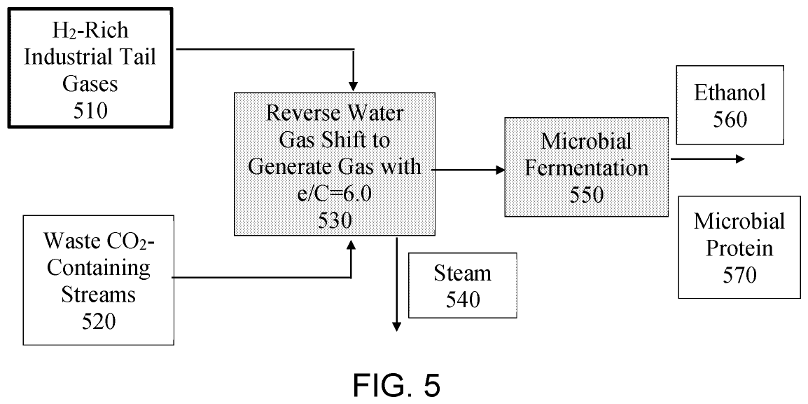

The process of producing ethanol by microbial fermentation by reforming with hydrogen rich industrial tail gas and waste CO₂-containing streams such as acid gas (carbon fixing by reverse water gas shift) is set forth in FIG. 5. Reverse water gas shift refers to moving the reversible water gas shift reaction balance backward and results in higher CO concentration in the equilibrium because of high H₂ and CO₂ content in the starting balance with high temperature. As seen in FIG. 5, H₂-rich industrial tail gases 510 are mixed with waste CO₂ containing streams 520 and undergo reverse water gas shift to generate gas 530 with e/C of 6.0. Steam 540 is released. The gas undergoes microbial fermentation 550 in accordance with embodiments of the disclosure and broth is removed from the reactor and ethanol 560 is recovered by techniques such as distillation. Biosolids enriched in microbial protein 570 are also recovered from the removed broth.

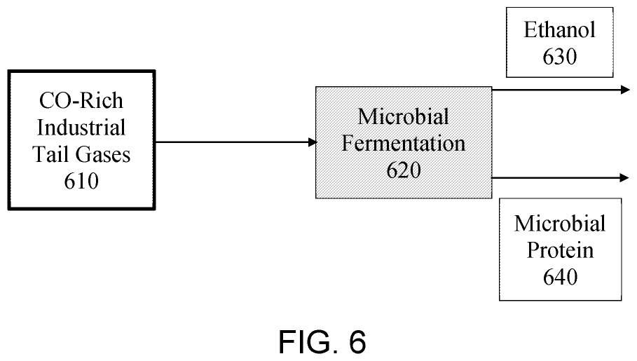

Ethanol can be produced by microbial fermentation using CO-rich industrial tail gases, such as acetic acid purge gas, calcium carbide furnace fail gas, steel mill gas, etc. The process of producing ethanol by microbial fermentation by direct feed to fermentation of carbon monoxide rich industrial tail gas is set forth in FIG. 6. As seen in FIG. 6, CO-rich industrial tail gases 610 undergo microbial fermentation 620 in accordance with embodiments of the disclosure. Broth is removed from the reactor and ethanol 630 is recovered by techniques such as distillation. Biosolids enriched in microbial protein 640 are also recovered from the removed broth.

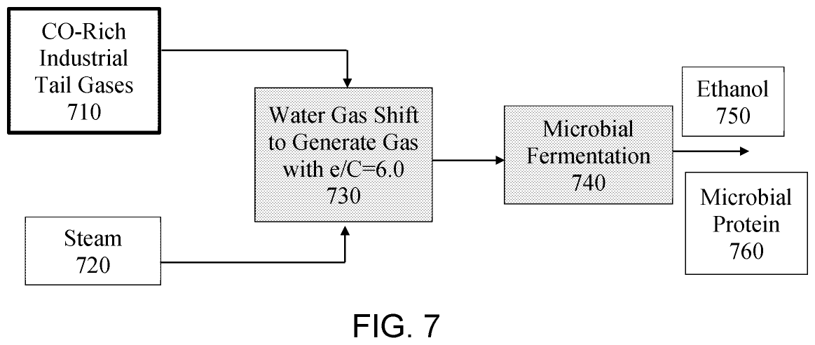

A representative process of producing ethanol by microbial fermentation by reforming carbon monoxide rich industrial tail gas by water gas shift is set forth in FIG. 7. Water gas shift refers to converting CO and water vapor to H₂ and CO₂ and results in higher H₂ concentration in the equilibrium. The reverse of the water gas shift reaction is called ‘reverse water gas shift,’ where CO₂ and H₂ react to form CO and H₂O. In this regard, adding H₂ for a reverse water gas shift would not directly increase H₂ if it is all consumed in the reaction. The amount of CO₂ will decrease as a result of the reverse water gas shift reaction, and optionally if excess H₂ is added, the H₂ increases via that addition such that the overall relative amount of hydrogen increases. As seen in FIG. 7, CO-rich industrial tail gases 710 are combined with steam 720 and undergo water gas shift to generate gas with e/C of, e.g., at least about 5.7 (such as from about 5.7 to about 8.0) 730. The gas undergoes microbial fermentation 740 in accordance with embodiments of the disclosure. Broth is removed from the reactor and ethanol 750 is recovered by techniques such as distillation. Biosolids enriched in microbial protein 760 are also recovered from the removed broth.

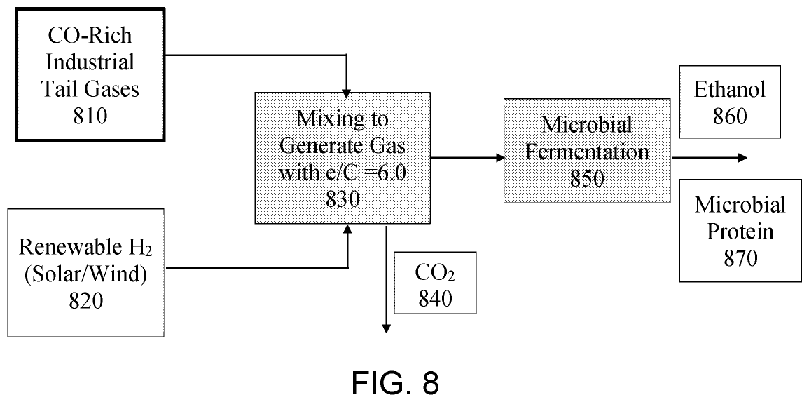

The process of producing ethanol by microbial fermentation by mixing with renewable H₂ (carbon fixing with renewable H₂) is set forth in FIG. 8. As seen in FIG. 8, CO-rich industrial tail gases 810 are combined with renewable H₂ (solar/wind) 820 and are mixed to generate gas 830 with e/C of, e.g., at least about 5.7 (such as from about 5.7 to about 8.0). CO₂ 840 is released. The gas undergoes microbial fermentation 850 in accordance with embodiments of the disclosure. Broth is removed from the reactor and ethanol 860 is recovered by techniques such as distillation. Biosolids enriched in microbial protein 870 are also recovered from the removed broth.

### Renewable Sources of Hydrogen Gas

The syngas can be enriched with hydrogen gas to form a H₂-enriched syngas that is derived at least in part from “green” technology. The syngas can be blended with hydrogen in any suitable manner and from any suitable source to prepare the H₂-enriched syngas which is in turn fermented as described herein.

In accordance with embodiments of the disclosure, industrial purge gases are repurposed to produce the hydrogen enriched syngas. Additionally, in some embodiments, hydrogen gas produced by environmentally-friendly, renewable sources (e.g., wind, solar, or a combination thereof) can be used to enrich the syngas with hydrogen gas. Surprisingly and unexpectedly, the present inventors have discovered that the process can beneficially avoid the use of a water gas shift reaction, which undesirably forms excess CO₂ that would have to be mitigated.

In this respect, water gas shift has been used conventionally to improve hydrogen content in syngas. For example, a conventional problem with biomass, MSW, or coal-based syngas is that it has an elevated CO content and a relatively low hydrogen content, which complicates a number of processes. Traditionally, in order to circumvent the issue, the water gas shift reaction is employed to increase the hydrogen content of syngas at the expense of the conversion of CO to CO₂. In this respect, the water gas shift reaction refers to converting CO and water vapor to H₂ and CO₂ and results in higher H₂ concentration in the equilibrium.

The water gas shift reaction is an exothermic reaction between carbon monoxide and steam to form carbon dioxide and hydrogen. Generally, in typical industrial applications, the water gas shift reaction is conducted as a two-stage process. The stages are conventionally split between a “high temperature” stage and a “low temperature” stage. The high temperature stage is conducted over an iron based catalyst in a range of about 320-450° C. The low temperature stage is conducted over copper-based catalysts in a range of about 150-250° C.

Use of the water gas shift reaction results in increased levels of hydrogen; however, amounts of CO₂ are also inevitably produced. CO₂ is a greenhouse gas, and limited options exist for its capture and use. If all the CO₂ produced by the water gas shift reaction is not consumed, the process risks becoming a net CO₂ producer. As such, there is a need to mitigate surplus CO₂ via additional processes (e.g., carbon capture), thereby introducing further complexity and steps to the process.

In accordance with embodiments of the disclosure, the inventors have found that water gas shift techniques can be avoided by directly adjusting the amount of hydrogen through the use of renewable hydrogen. By way of this process, the water gas shift reaction can be avoided since the addition of renewable hydrogen enables the specific adjustments of the hydrogen content. This enables adjusting the amount of hydrogen to a tolerable range without the use of water gas shift reaction which generally produces surplus CO₂. Importantly, unlike previous uses of renewable for the conversion of syngas (Wang et al.) use of renewably hydrogen enhanced syngas for fermentation via carboxytrophs does not require the removal of H₂S or CO₂ from the enhanced syngas stream. In fact, the H₂S enhances the efficiency of the process, in that it can be used by the homoacetogenic carboxytrophs to help offset the need for supplemental sources of sulfur.

In some embodiments, the renewable hydrogen is added to syngas formed from waste feedstocks, e.g., MSW. MSW is a readily available and easily sourced feedstock as it is generally buried or incinerated if not otherwise used. The incineration of MSW results in the release of CO₂ and particulates (e.g., soot), while burial enables the microbial conversion of MSW, which releases “biogas” as a result. Biogas is a mixture of H₂S, CO₂, and methane (CH₄). As described herein, CO₂ is a pollutant, H₂S is flammable, corrosive and poisonous, and CH₄ is considered a more dangerous greenhouse gas than CO₂. Preparing syngas formed from biomass, e.g., in the form of MSW, can desirably prevent the release of such pollutants and particulates that would otherwise be released by means of burial and/or incineration.

Gasification of MSW typically results in a syngas with a H₂:CO ratio closer to 1:1 (with an e/C of approximately ≤3), in keeping with most substrates for gasification (e.g., coal and biomass) which also result in H₂:CO ratios near 1:1. In this respect, syngas prepared from MSW would need its H₂ content enhanced to be considered desirably efficient for ethanol production.

Any suitable amount of hydrogen can be added to the syngas to form the H₂-enriched syngas. For example, in some embodiments, the enriching comprises adding H₂ from the renewable source to the syngas to increase the amount of the H₂ to at least about 50 vol. %, e.g., from about 50 vol. % to about 70 vol. %, or from about 60 vol. % to about 70 vol. % of H₂, in the H₂-enriched syngas.

In some embodiments, the syngas is blended with the hydrogen gas to prepare an H₂-enriched syngas characterized by an e/C to a value of at least about 5.7, e.g., to a value of from about 5.7 to about 8.0.

The production of the hydrogen gas, according to embodiments of the disclosure, can be from any renewable source. For example, the renewable source can be in the form of a solar panel array or farm containing wind turbines, or a combination thereof. In general, the renewable source can produce electricity which can then be transmitted to a location where an electrolysis process is carried out to convert water to hydrogen and oxygen. The hydrogen can be delivered to the location of the syngas production by way of, e.g., hydrogen piping, hydrogen liquefaction and tank car transportation, and other hydrogen storage and transportation technologies.

Generally, sources such as solar panels and wind turbines can be used as renewable sources of electricity. The wind and solar power can be produced in any suitable manner using known techniques. For example, onshore or offshore wind turbines can be used via propeller-like blades of the turbine around a rotor. The blades of the turbine create an aerodynamic force that causes the rotor to spin. A generator converts the mechanical (kinetic) energy of the rotor to electrical energy. In the case of solar technology, sunlight is converted into electrical energy in any suitable manner, such as photovoltaic panels, or by using mirrors that concentrate solar radiation, etc. The energy creates electric charges that move in response to an internal electrical field in the cell, thereby allowing electricity to flow. Techniques for forming electricity from renewable sources are well known in the art, and any suitable technique or arrangement for renewably forming electricity can be used in accordance with embodiments of the disclosure. See, e.g., U.S. Pat. Nos. 2,360,791 A; 7,709,730 B2; 7,381,886 B1; 7,821,148 B2; 8,866,334 B2; 9,871,255 B2; 9,938,627 B2; and 2022/0145479 A1.

In some embodiments, the electricity used in methods according to the disclosure can have its renewability documented and desirably be designated as “clean” power by appropriate bodies. For renewable energy, preferably, the same amount of power is returned to the grid as is being used. Since water is desirably considered renewable, when used with renewable power, then the produced hydrogen is considered renewable, too, in accordance with some embodiments of the present disclosure.

Once sourced, the electricity is used to produce hydrogen, e.g., through electrolysis, which splits water into the desired hydrogen as well as oxygen. This method allows for the production of renewable hydrogen via electrolysis where the hydrogen is used to enrich the syngas so that it can be used to produce an oxygenated product (such as ethanol) without the use of water gas shift and the requirement to mitigate its surplus CO₂ production.

In accordance with embodiments of the disclosure, the inventors have found that water gas shift techniques can be avoided by directly adjusting the amount of hydrogen via the use of renewable hydrogen. The addition of renewable hydrogen enables specific adjustment of the amount of hydrogen to a tolerable range without the use of water gas shift reaction and without producing surplus CO₂. In addition, in accordance with embodiments, the need for additional steps to ensure the removal of components such as H₂S and CO₂ from the syngas is unnecessary. H₂S, for example, can negatively affect the production of methanol by catalytic routes, but, in accordance with embodiments, do not negatively affect the process as disclosed herein. Specifically, the presence of H₂S can be used as a source of sulfur for the organism, thereby desirably reducing costs and labor associated with the process. Advantageously, as a result, syngas is formed that is more suitable for producing oxygenated products such as ethanol with fewer steps and hurdles in the process.

### H₂ Enriched Syngas

In accordance with embodiments of the disclosure, the syngas is blended with an industrial purge gas and/or hydrogen gas from a renewable source to form the H₂-enriched syngas. As a result, the hydrogen content and/or e/C in the H₂-enriched syngas is higher than the hydrogen content and/or e/C in the syngas alone. In some embodiments, the H₂-rich tail gas is derived from purge gas from a coal derived chemical production process, such as purge gas from coal to methanol production, purge gas from coal to synthetic ammonia production, purge gas from coal to acetic acid production, purge gas from coal to ethylene glycol production, purge gas from coal to synthetic natural gas production, purge gas from coal to liquid production, coke oven gas, or any combination thereof.

The H₂-enriched syngas can generally have any suitable hydrogen content, although the hydrogen content will be greater in the H₂-enriched syngas on a relative volume basis as compared with the syngas. For example, in some embodiments, the H₂-enriched syngas contains at least about 50 vol. % of H₂, e.g., from about 50 vol. % to about 85 vol. %, or from about 60 vol. % to about 70 vol. % of H₂.

Typically, the H₂-enriched syngas has an e/C of at least about 5.7. In some embodiments, the H₂-enriched syngas has an e/C of about 8 or less, e.g., from about 5.7 to about 8.0. In this regard, the H₂-enriched syngas normally will have a higher e/C as compared with the syngas because of higher H₂ content in the H₂-enriched syngas.

The H₂-enriched syngas can generally have any suitable carbon monoxide content. For example, in some embodiments, the H₂-enriched syngas contains from about 3 vol. % to about 50 vol. % of CO, e.g., from about 25 vol. % to about 35 vol. % of CO. In some embodiments, the H₂-enriched syngas will have a lower relative volume percentage of carbon monoxide as compared with the syngas without hydrogen enrichment.

The H₂-enriched syngas can generally have any suitable carbon dioxide content. For example, in some embodiments, the syngas contains from about 0 vol. % to about 15 vol. % of CO₂, e.g., from about 3 vol. % to about 15 vol. % of CO₂ or from about 3 vol. % to about 5 vol. % of CO₂. In some embodiments, the H₂-enriched syngas will have a lower relative volume percentage of carbon dioxide as compared with the syngas.

### Microorganism

Any suitable microorganism can be used for the fermentation in the methods of the disclosure, e.g., bacteria that are well suited to ferment gases containing higher contents of hydrogen gases (e.g., containing at least about 50% by volume of hydrogen gas). For example, in some embodiments, the bacteria are acetogenic carboxydotrophs. Such microorganisms are described in commonly-assigned co-pending U.S. Application Nos. 63/136,025 and 63/136,042, which are hereby incorporated by reference.

For example, in some embodiments, the microorganisms used in fermentation in the methods of the disclosure are in the form of bacteria comprising Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof. These bacteria are characterized by the presence of a Wood-Ljungdahl metabolic pathway, as discussed in U.S. Pat. No. 6,340,581 B1.

### Oxygenated Product

As described herein, upon fermentation, the microorganism produces an oxygenated product in embodiments of the disclosure. The oxygenated product can be recovered from the broth by any suitable technique, including, but not limited to, fractional distillation, evaporation, pervaporation, gas stripping, phase separation, and extractive fermentation, including for example, liquid-liquid extraction, or any combination thereof.

Any suitable oxygenated product as desired that can be prepared from the methods described herein can be produced. For example, in some embodiments, the oxygenated product is ethanol. In some embodiments, the oxygenated product is acetic acid, butyrate, butanol, propionate, propanol, or any combination thereof. In some embodiments, the method further comprises separating the oxygenated product from the broth.

The production of a particularly desired oxygenated product can be achieved using the fermentation process, as will be appreciated by one of ordinary skill in the art. For example, acetogenic carboxydotroph microorganisms can make acetate in their natural state, but conditions can be manipulated to make ethanol. By way of example, the pH of the fermentation broth can be reduced to about 5.3 or less (such as about 4.8 or less) and the amount of vitamin B5 can be limited to thereby constrain growth of microorganism and allow for production of more ethanol. Other oxygenated compounds, such as propionate, butyrate, acetic acid, butanol, and propanol, can be made by using alternative carboxytrophic organism, engineering the acetogenic carboxydotroph microorganisms (see, e.g., U.S. Patent Publication No. 2011/0236941 A1), by the use of co-cultures (see, e.g., U.S. Pat. No. 9,469,860 B2 and U.S. Patent Publication No. 2014/0273123 A1) or addition or modification of components as will be within the level of skill of one of ordinary skill in the art.

### Co-Localization

While not required, co-localization can be used in some embodiments in the production process for forming the oxygenated and/or feed product. As used herein, co-localization can involve the use of renewable hydrogen but it is not limited as such. Co-localization includes locating different component processes in one centralized area at a single site or in close proximity to each other (e.g., within about 50 miles, such as within about 10 miles or about 5 miles). For example, this may include locating the syngas production, production of purge (tail) gas, hydrogen enrichment of syngas, fermentation, electrolysis (if present), electricity production (if present, e.g., by means of solar and/or wind), and/or separation of oxygenated product, at one site or in close proximity to each other.

In embodiments, the syngas production, production of purge (tail) gas, hydrogen enrichment of syngas, fermentation, and/or separation of oxygenated product processes can be co-localized in any suitable arrangement. For example, in embodiments, the syngas production and the production of purge (tail) gas processes are co-localized. In embodiments, the syngas production and the hydrogen enrichment of syngas processes are co-localized. In embodiments, the syngas production and the fermentation processes are co-localized. In embodiments, the syngas production and the separation of oxygenated product processes are co-localized. In embodiments, the production of purge (tail) gas and the hydrogen enrichment of syngas processes are co-localized. In embodiments, the production of purge (tail) gas and the fermentation processes are co-localized. In embodiments, the production of purge (tail) gas and the separation of oxygenated product processes are co-localized. In embodiments, the hydrogen enrichment of syngas and the fermentation processes are co-localized. In embodiments, the hydrogen enrichment of syngas and the separation of oxygenated product processes are co-localized. In embodiments, the fermentation and the separation of oxygenated product processes are co-localized.

In embodiments where renewable hydrogen is added to the syngas to form H₂-enriched syngas, the syngas production, hydrogen enrichment of syngas, fermentation, electrolysis, electricity production, and/or separation of oxygenated product processes can be co-localized in any suitable arrangement. For example, in embodiments, the syngas production and the hydrogen enrichment of syngas processes are co-localized. In embodiments, the syngas production and the fermentation processes are co-localized. In embodiments, the syngas production and the electrolysis processes are co-localized. In embodiments, the syngas production and the electricity production processes are co-localized. In embodiments, the syngas production and the separation of oxygenated product processes are co-localized. In embodiments, the hydrogen enrichment of syngas and the fermentation processes are co-localized. In embodiments, the hydrogen enrichment of syngas and the electrolysis processes are co-localized. In embodiments, the hydrogen enrichment of syngas and the electricity production processes are co-localized. In embodiments, the hydrogen enrichment of syngas and the separation of oxygenated product processes are co-localized. In embodiments, the fermentation and the electrolysis processes are co-localized. In embodiments, the fermentation and the electricity production processes are co-localized. In embodiments, the fermentation and the separation of oxygenated product processes are co-localized. In embodiments, the electrolysis and the electricity production processes are co-localized. In embodiments, the electrolysis and the separation of oxygenated product processes are co-localized. In embodiments, the electricity production and the separation of oxygenated product processes are co-localized.

In embodiments, the syngas production, purge gas production, syngas enrichment with hydrogen, and fermentation processes are co-localized. In embodiments, the fermentation, electrolysis, syngas production, and syngas enrichment with hydrogen, as well as the source of electricity, are co-localized. In embodiments, the syngas production, hydrogen enrichment of syngas, fermentation process, and separation of oxygenated product are co-localized. In embodiments, all aspects of the production process are co-localized.

In some embodiments, the co-localization method involves sourcing electricity (e.g., from either a non-renewable or a renewable source) to generate the production of hydrogen using electrolysis. However, since electricity can be efficiently produced and transported over long distances through transmission lines, the electricity sourced in this process can either be produced on-site, in close proximity, or transported by transmission line and still be considered a co-localized process for making product in accordance with embodiments of the present disclosure. If desired, a direct transmission line can be used, e.g., in locations where maintenance of the plant's own grid is economically beneficial (e.g., over-taxed or unstable local grids susceptible to outages).

### Aspects

The invention is further illustrated by the following exemplary aspects. However, the invention is not limited by the following aspects.

(1) A method of preparing an oxygenated product, the method comprising: (a) providing a syngas comprising at least two of the following components: CO, CO₂, and H₂; (b) enriching the H₂ content in the syngas to form a H₂-enriched syngas; and (c) fermenting the H₂-enriched syngas with acetogenic carboxydotrophic bacteria in a liquid medium to form a broth in a bioreactor to produce an oxygenated product in the broth.

(2) The method of aspect 1, wherein the syngas contains from about 5 vol. % to about 80 vol. % of H₂, or from about 50 vol. % to about 80 vol. % of H₂.

(3) The method of aspects 1 or 2, wherein the syngas contains from about 3 vol. % to about 85 vol. % of CO, e.g., from about 10 vol. % to about 50 vol. % of CO.

(4) The method of any one of aspects 1 or 2, wherein the syngas contains from about 3 vol. % to about 45 vol. % of CO₂, e.g., from about 0 vol. % to about 25 vol. % of CO₂.

(5) The method of any one of aspects 1-4, wherein the syngas contains from about 0 vol. % to about 45 vol. % of CO₂, e.g., from about 3 vol. % to about 25 vol. % of CO₂.

(6) The method of any one of aspects 1-5, wherein the H₂-enriched syngas contains at least about 50 vol. % of H₂, e.g., from about 50 vol. % to about 85 vol. %, or from about 60 vol. % to about 70 vol. % of H₂.

(7) The method of any one of aspects 1-6, wherein the H₂-enriched syngas contains from about 3 vol. % to about 50 vol. % of CO, e.g., from about 25 vol. % to about 35 vol. % of CO.

(8) The method of any one of aspects 1-7, wherein the H₂-enriched syngas contains from about 3 vol. % to about 15 vol. % of CO₂, e.g., from about 0 vol. % to about 5 vol. % of CO₂.

(9) The method of any one of aspects 1-7, wherein the H₂-enriched syngas contains from about 3 vol. % to about 15 vol. % of CO₂, e.g., from about 3 vol. % to about 5 vol. % of CO₂.

(10) The method of any one of aspects 1-9, wherein the syngas has an e/C of at least about 2, e.g., from about 2 to about 8 or from about 2 to about 6.0.

(11) The method of any one of aspects 1-9, wherein the syngas has an e/C of at least about 2, e.g., from about 2 to about 8 or from about 2 to about 5.7.

(12) The method of any one of aspects 1-9, wherein the syngas has an e/C of at least about 2, e.g., from about 2 to about 6 or from about 2 to about 5.7.

(13) The method of any one of aspects 1-12, wherein the H₂-enriched syngas has an e/C of about 6 or less, e.g., from about 5.7 to about 6.

(14) The method of any one of aspects 1-13, wherein the oxygenated product is ethanol.

(15) The method of any one of aspects 1-14, wherein the oxygenated product is acetic acid, butyrate, butanol, propionate, propanol, or any combination thereof.

(16) The method of any one of aspects 1-15, the method further comprising separating the oxygenated product from the broth.

(17) The method of aspect 16, wherein the oxygenated product is separated by fractional distillation, evaporation, pervaporation, gas stripping, phase separation, and extractive fermentation, including for example, liquid-liquid extraction, or any combination thereof.

(18) The method of any one of aspects 1-17, wherein the bacteria comprises Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

(19) The method of any one of aspects 1-18, wherein the enriching comprises mixing the syngas with H₂-rich tail gas.

(20) The method of aspect 19, wherein the H₂-rich tail gas contains at least about 50 vol. % of H₂, e.g., from about 50 vol. % to about 85 vol. %, or from about 60 vol. % to about 70 vol. % of H₂.

(21) The method of aspects 18 or 19, wherein the H₂-rich tail gas is derived from purge gas from a coal derived chemical production process, such as purge gas from coal to methanol production, purge gas from coal to synthetic ammonia production, purge gas from coal to acetic acid production, purge gas from coal to ethylene glycol production, purge gas from coal to synthetic natural gas production, purge gas from coal to liquid production, coke oven gas, or any combination thereof.

(22) The method of any one of aspects 1-18, wherein the syngas contains at least about 15 vol. % of CO₂, and the enriching comprises adding H₂-rich industrial tail gas and steam to the syngas to effect a reverse water gas shift to increase the e/C to a value of from about 5.7 to about 6.

(23) The method of any one of aspects 1-18, wherein the syngas contains at least about 15 vol. % of CO₂, and the enriching comprises adding H₂-rich industrial tail gas to the syngas to effect a reverse water gas shift reaction to convert CO₂ to CO and optionally excess H₂ added to increase the amount of the H₂ to at least about 50 vol. %, e.g., from about 50 vol. % to about 70 vol. %, or from about 60 vol. % to about 70 vol. % of H₂.

(24) The method of any one of aspects 1-18, wherein the syngas contains at least about 35 vol. % of CO, and the enriching comprises adding steam to the syngas to effect a water gas shift to increase the e/C to a value of from about 5.7 to about 6.

(25) The method of any one of aspects 1-18, wherein the syngas contains at least about 35 vol. % of CO, and the enriching comprises adding steam to the syngas to effect a water gas shift to increase the amount of the H₂ to at least about 50 vol. %, e.g., from about 50 vol. % to about 70 vol. %, or from about 60 vol. % to about 70 vol. % of H₂.

(26) The method of any one of aspects 1-18, wherein the syngas contains at least about 35 vol. % of CO, and the enriching comprises adding H₂ from a renewable source to the syngas to increase the e/C to a value of from about 5.7 to about 6.

(27) The method of any one of aspects 1-18, wherein the syngas contains at least about 35 vol. % of CO, and the enriching comprises adding H₂ from a renewable source to the syngas to increase the amount of the H₂ to at least about 50 vol. %, e.g., from about 50 vol. % to about 70 vol. %, or from about 60 vol. % to about 70 vol. % of H₂.

(28) The method of any one of aspects 1-27, wherein the syngas is coal derived syngas.

(29) The method of aspects 26 or 27, wherein the renewable source for the H₂ is solar, wind, or a combination thereof, e.g., renewable source (namely the sun or wind) generates electricity to run electrolysis to produce renewable hydrogen.

(30) A method of preparing an oxygenated product, the method comprising: (a) providing a syngas comprising at least two of the following components: CO, CO₂, and H₂; (b) enriching the H₂ content in the syngas to form a H₂-enriched syngas having at least about 50 vol. % of H₂, e.g., from about 50 vol. % to about 85 vol. %, from about 50 vol. % to about 70 vol. %, or from about 60 vol. % to about 70 vol. % of H₂; (c) fermenting the H₂-enriched syngas with bacteria in a liquid medium to form a broth in a bioreactor to produce an oxygenated product in the broth.

(31) The method of aspect 30, wherein the syngas contains from about 5 vol. % to about 80 vol. % of H₂, or from about 50 vol. % to about 80 vol. % of H₂.

(32) The method of aspects 30 or 31, wherein the syngas contains from about 3 vol. % to about 85 vol. % of CO, e.g., from about 10 vol. % to about 50 vol. % of CO.

(33) The method of any one of aspects 30-32, wherein the syngas contains from about 3 vol. % to about 45 vol. % of CO₂, e.g., from about 0 vol. % to about 25 vol. % of CO₂.

(34) The method of any one of aspects 30-32, wherein the syngas contains from about 3 vol. % to about 45 vol. % of CO₂, e.g., from about 3 vol. % to about 25 vol. % of CO₂.

(35) The method of any one of aspects 30-34, wherein the H₂-enriched syngas contains from about 3 vol. % to about 50 vol. % of CO, e.g., from about 25 vol. % to about 35 vol. % of CO.

(36) The method of any one of aspects 30-35, wherein the H₂-enriched syngas contains from about 0 vol. % to about 15 vol. % of CO₂, e.g., from about 3 vol. % to about 5 vol. % of CO₂.

(37) The method of any one of aspects 30-36, wherein the syngas has an e/C of at least about 2, e.g., from about 2 to about 8.

(38) The method of any one of aspects 30-36, wherein the syngas has an e/C of at least about 2, e.g., from about 2 to about 6.

(39) The method of any one of aspects 30-38, wherein the H₂-enriched syngas has an e/C of about 6 or less, e.g., from about 5.7 to about 6.

(40) The method of any one of aspects 30-39, wherein the oxygenated product is ethanol.

(41) The method of any one of aspects 30-40, wherein the oxygenated product is acetic acid, butyrate, butanol, propionate, propanol, or any combination thereof.

(42) The method of any one of aspects 30-41, the method further comprising separating the oxygenated product from the broth.

(43) The method of aspect 42, wherein the oxygenated product is separated by fractional distillation, evaporation, pervaporation, gas stripping, phase separation, and extractive fermentation, including for example, liquid-liquid extraction, or any combination thereof.

(44) The method of any one of aspects 30-43, wherein the bacteria comprises Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

(45) The method of any one of aspects 30-44, wherein the enriching comprises mixing the syngas with H₂-rich tail gas.

(46) The method of aspect 45, wherein the H₂-rich tail gas contains at least about 50 vol. % of H₂, e.g., from about 50 vol. % to about 85 vol. %, or from about 60 vol. % to about 70 vol. % of H₂.

(47) The method of aspects 45 or 46, wherein the H₂-rich tail gas is derived from purge gas from a coal derived chemical production process, such as purge gas from coal to methanol production, purge gas from coal to synthetic ammonia production, purge gas from coal to acetic acid production, purge gas from coal to ethylene glycol production, purge gas from coal to synthetic natural gas production, purge gas from coal to liquid production, coke oven gas, or any combination thereof.

(48) The method of any one of aspects 30-44, wherein the syngas contains at least about 15 vol. % of CO₂, and the enriching comprises adding H₂-rich industrial tail gas and steam to the syngas to effect a reverse water gas shift to increase the e/C to a value of from about 5.7 to about 6.

(49) The method of any one of aspects 30-44, wherein the syngas contains at least about 0 vol. % of CO₂, and the enriching comprises adding H₂-rich industrial tail gas and steam to the syngas to effect a reverse water gas shift to increase the e/C to a value of from about 5.7 to about 6.

(50) The method of any one of aspects 30-44, wherein the syngas contains at least about 15 vol. % of CO₂, and the enriching comprises adding H₂-rich industrial tail gas to the syngas to effect a reverse water gas shift reaction to convert CO₂ to CO and optionally excess H₂ added to increase the amount of the H₂ to at least about 50 vol. %, e.g., from about 50 vol. % to about 70 vol. %, or from about 60 vol. % to about 70 vol. % of H₂.

(51) The method of any one of aspects 30-44, wherein the syngas contains at least about 35 vol. % of CO and the enriching comprises adding steam to the syngas to effect a water gas shift to increase the e/C to a value of from about 5.7 to about 6.

(52) The method of any one of aspects 30-44, wherein the syngas contains at least about 35 vol. % of CO and the enriching comprises adding steam to the syngas to effect a water gas shift to increase the amount of the H₂ to at least about 50 vol. %, e.g., from about 50 vol. % to about 70 vol. %, or from about 60 vol. % to about 70 vol. % of H₂.

(53) The method of any one of aspects 30-44, wherein the syngas contains at least about 35 vol. % of CO and the enriching comprises adding H₂ from a renewable source to the syngas to increase the e/C to a value of from about 5.7 to about 6.

(54) The method of any one of aspects 30-44, wherein the syngas contains at least about 35 vol. % of CO and the enriching comprises adding H₂ from a renewable source to the syngas to increase the amount of the H₂ to at least about 50 vol. %, e.g., from about 50 vol. % to about 70 vol. %, or from about 60 vol. % to about 70 vol. % of H₂.

(55) The method of aspects 53 or 54, wherein the renewable source for H₂ is solar, wind, or a combination thereof, e.g., renewable source (namely the sun or wind) generates electricity to run electrolysis to produce renewable hydrogen.

(56) The method of any one of aspects 30-54, wherein the syngas is coal derived syngas.

(57) The method of any one of aspects 30-56, wherein the bacteria is an acetogenic carboxydotroph.

(58) A method of preparing an oxygenated product, the method comprising: (a) providing a syngas comprising at least two of the following components: CO, CO₂, and H₂; (b) enriching the H₂ content in the syngas to form a H₂-enriched syngas having an e/C of at least about 5.7, e.g., from about 5.7 to about 8; (c) fermenting the H₂-enriched syngas with bacteria in a liquid medium to form a broth in a bioreactor to produce an oxygenated product in the broth.

(59) A method of preparing an oxygenated product, the method comprising: (a) providing a syngas comprising at least two of the following components: CO, CO₂, and H₂; (b) enriching the H₂ content in the syngas to form a H₂-enriched syngas having an e/C of at least about 5.7, e.g., from about 5.7 to about 6; (c) fermenting the H₂-enriched syngas with bacteria in a liquid medium to form a broth in a bioreactor to produce an oxygenated product in the broth.

(60) The method of aspect 58, wherein the syngas contains from about 5 vol. % to about 80 vol. % of H₂, or from about 50 vol. % to about 80 vol. % of H₂.

(61) The method of aspects 58 or 60, wherein the syngas contains from about 3 vol. % to about 85 vol. % of CO, e.g., from about 10 vol. % to about 50 vol. % of CO.

(62) The method of any one of aspects 58-61, wherein the syngas contains from about 0 vol. % to about 45 vol. % of CO₂, e.g., from about 3 vol. % to about 25 vol. % of CO₂.

(63) The method of any one of aspects 58-61, wherein the syngas contains from about 3 vol. % to about 45 vol. % of CO₂, e.g., from about 0 vol. % to about 25 vol. % of CO₂.

(64) The method of any one of aspects 58-63, wherein the H₂-enriched syngas contains at least about 50 vol. % of H₂, e.g., from about 50 vol. % to about 85 vol. %, or from about 60 vol. % to about 70 vol. % of H₂.

(65) The method of any one of aspects 58-64, wherein the H₂-enriched substrate gas contains from about 3 vol. % to about 50 vol. % of CO, e.g., from about 25 vol. % to about 35 vol. % of CO.

(66) The method of any one of aspects 58-65, wherein the H₂-enriched syngas contains from about 0 vol. % to about 15 vol. % of CO₂, e.g., from about 3 vol. % to about 5 vol. % of CO₂.

(67) The method of any one of aspects 58-65, wherein the H₂-enriched syngas contains from about 0 vol. % to about 15 vol. % of CO₂, e.g., from about 3 vol. % to about 5 vol. % of CO₂.

(68) The method of any one of aspects 58-67, wherein the syngas has an e/C of at least about 2, e.g., from about 2 to about 6.

(69) The method of any one of aspects 58-68, wherein the oxygenated product is ethanol.

(70) The method of any one of aspects 58-69, wherein the oxygenated product is acetic acid, butyrate, butanol, propionate, propanol, or any combination thereof.

(71) The method of any one of aspects 58-70, the method further comprising separating the water from the oxygenated product.

(72) The method of aspect 71, wherein the oxygenated product is separated by fractional distillation, evaporation, pervaporation, gas stripping, phase separation, and extractive fermentation, including for example, liquid-liquid extraction, or any combination thereof.

(73) The method of any one of aspects 58-72, wherein the bacteria comprises Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

(74) The method of any one of aspects 58-73, wherein the enriching comprises mixing the syngas with H₂-rich tail gas.

(75) The method of aspect 74, wherein the H₂-rich tail gas contains at least about 50 vol. % of H₂, e.g., from about 50 vol. % to about 85 vol. %, or from about 60 vol. % to about 70 vol. % of H₂.

(76) The method of aspects 74 or 75, wherein the H₂-rich tail gas is derived from purge gas from a coal derived chemical production process, such as purge gas from coal to methanol production, purge gas from coal to synthetic ammonia production, purge gas from coal to acetic acid production, purge gas from coal to ethylene glycol production, purge gas from coal to synthetic natural gas production, purge gas from coal to liquid production, coke oven gas, or any combination thereof.

(77) The method of any one of aspects 58-73, wherein the syngas contains at least about 15 vol. % of CO₂, and the enriching comprises adding H₂-rich industrial tail gas and steam to the syngas to effect a reverse water gas shift to increase the e/C to a value of from about 5.7 to about 6.

(78) The method of any one of aspects 58-73, wherein the syngas contains at least about 0 vol. % of CO₂, and the enriching comprises adding H₂-rich industrial tail gas and steam to the syngas to effect a reverse water gas shift to increase the e/C to a value of from about 5.7 to about 6.

(79) The method of any one of aspects 71-73, wherein the syngas contains at least about 15 vol. % of CO₂, and the enriching comprises adding H₂-rich industrial tail gas to the syngas to effect a reverse water gas shift to increase the amount of the H₂ to at least about 50 vol. %, e.g., from about 50 vol. % to about 70 vol. %, or from about 60 vol. % to about 70 vol. % of H₂.

(80) The method of any one of aspects 71-73, wherein the syngas contains at least about 0 vol. % of CO₂, and the enriching comprises adding H₂-rich industrial tail gas to the syngas to effect a reverse water gas shift to increase the amount of the H₂ to at least about 50 vol. %, e.g., from about 50 vol. % to about 70 vol. %, or from about 60 vol. % to about 70 vol. % of H₂.

(81) The method of any one of aspects 58-73, wherein the syngas contains at least about 35 vol. % of CO and the enriching comprises adding steam to the syngas to effect a water gas shift to increase the e/C to a value of from about 5.7 to about 6.

(82) The method of any one of aspects 58-73, wherein the syngas contains at least about 35 vol. % of CO and the enriching comprises adding steam to the syngas to effect a water gas shift to increase the amount of the H₂ to at least about 50 vol. %, e.g., from about 50 vol. % to about 70 vol. %, or from about 60 vol. % to about 70 vol. % of H₂.

(83) The method of any one of aspects 58-73, wherein the syngas contains at least about 35 vol. % of CO and the enriching comprises adding H₂ from a renewable source to the syngas to increase the e/C to a value of from about 5.7 to about 8.

(84) The method of any one of aspects 58-73, wherein the syngas contains at least about 35 vol. % of CO and the enriching comprises adding H₂ from a renewable source to the syngas to increase the e/C to a value of from about 5.7 to about 6.

(85) The method of any one of aspects 58-73, wherein the syngas contains at least about 35 vol. % of CO and the enriching comprises adding H₂ from a renewable source to the syngas to increase the amount of the H₂ to at least about 50 vol. %, e.g., from about 50 vol. % to about 70 vol. %, or from about 60 vol. % to about 70 vol. % of H₂.

(86) The method of aspects 83 or 84, wherein the renewable source for H₂ is solar, wind, or a combination thereof, e.g., renewable source (namely the sun or wind) generates electricity to run electrolysis to produce renewable hydrogen.

(87) The method of any one of aspects 58-73, wherein the syngas is coal derived syngas.

(88) The method of any one of aspects 58-87, wherein the bacteria is an acetogenic carboxydotroph.

(89) A method of renewably preparing an oxygenated product, the method comprising: (a) providing a syngas comprising at least two of the following compounds: CO, CO₂, and H₂; (b) adding H₂ from a renewable source to the syngas to form an H₂-enriched syngas; (c) fermenting the H₂-enriched syngas with bacteria in a liquid medium to form a broth in a bioreactor to produce an oxygenated product in the broth.

(90) The method of aspect 89, wherein the bacteria is an acetogenic carboxydotroph.

(91) The method of aspect 90, wherein the syngas contains from about 5 vol. % to about 80 vol. % of H₂, or from about 50 vol. % to about 80 vol. % of H₂.

(92) The method of any one of aspects 89-91, wherein the syngas contains from about 3 vol. % to about 85 vol. % of CO, e.g., from about 10 vol. % to about 50 vol. % of CO.

(93) The method of any one of aspects 89-92, wherein the syngas contains from about 0 vol. % to about 45 vol. % of CO₂, e.g., from about 3 vol. % to about 25 vol. % of CO₂.

(94) The method of any one of aspects 89-92, wherein the syngas contains from about 3 vol. % to about 45 vol. % of CO₂, e.g., from about 3 vol. % to about 25 vol. % of CO₂.

(95) The method of any one of aspects 89-94, wherein the H₂-enriched syngas contains at least about 50 vol. % of H₂, e.g., from about 50 vol. % to about 85 vol. %, or from about 60 vol. % to about 70 vol. % of H₂.

(96) The method of any one of aspects 89-95, wherein the H₂-enriched syngas contains from about 3 vol. % to about 50 vol. % of CO, e.g., from about 25 vol. % to about 35 vol. % of CO.

(97) The method of any one of aspects 89-96, wherein the H₂-enriched syngas contains from about 0 vol. % to about 15 vol. % of CO₂, e.g., from about 3 vol. % to about 5 vol. % of CO₂.

(98) The method of any one of aspects 89-96, wherein the H₂-enriched syngas contains from about 0 vol. % to about 15 vol. % of CO₂, e.g., from about 3 vol. % to about 5 vol. % of CO₂.

(99) The method of any one of aspects 89-98, wherein the syngas has an e/C of at least about 2, e.g., from about 2 to about 6.

(100) The method of any one of aspects 89-99, wherein the H₂-enriched syngas has an e/C of about 6 or less, e.g., from about 5.7 to about 6.

(101) The method of any one of aspects 89-100, wherein the oxygenated product is ethanol.

(102) The method of any one of aspects 89-101, wherein the oxygenated product is acetic acid, butyrate, butanol, propionate, propanol, or any combination thereof.

(103) The method of any one of aspects 89-102, the method further comprising separating the oxygenated product from the broth.

(104) The method of aspect 103, wherein the oxygenated product is separated by fractional distillation, evaporation, pervaporation, gas stripping, phase separation, and extractive fermentation, including for example, liquid-liquid extraction, or any combination thereof.

(105) The method of any one of aspects 89-104, wherein the bacteria comprises Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

(106) The method of any one of aspects 89-105, wherein the renewable source for H₂ is solar, wind, or any combination thereof, e.g., renewable source (namely the sun or wind) generates electricity to run electrolysis to produce renewable hydrogen.

(107) The method of any one of aspects 89-106, wherein the syngas contains at least about 35 vol. % of CO, and the adding of H₂ to the syngas increases the e/C to a value of from about 5.7 to about 6.

(108) The method of any one of aspects 89-107, wherein the syngas contains at least about 35 vol. % of CO, and the adding of H₂ to the syngas increases the amount of the H₂ to at least about 50 vol. %, e.g., from about 50 vol. % to about 70 vol. %, or from about 60 vol. % to about 70 vol. % of H₂.

(109) The method of any one of aspects 89-108, wherein the syngas is coal derived syngas.

(110) A method of preparing an animal feed, the method comprising: (a) providing a syngas comprising at least two of the following components: CO, CO₂, and H₂; (b) enriching the H₂ content in the syngas to form a H₂-enriched syngas, e.g., (i) to at least about 50 vol. % of H₂, such as from about 50 vol. % to about 85 vol. %, from about 50 vol. % to about 70 vol. % or from about 60 vol. % to about 70 vol. % of H₂, and/or (ii) to an e/C of at least about 5.7, such as from about 5.7 to about 6; (c) fermenting the H₂-enriched syngas with bacteria, such as acetogenic carboxydotrophic bacteria, in a liquid medium to form a broth in a bioreactor to produce an oxygenated product and a solid byproduct in the broth; (d) removing the oxygenated product from the broth to produce an oxygenated product-depleted broth; and (e) removing the solid byproduct from the broth and/or the oxygenated product-depleted broth to produce a cake and a clarified stream filtrate, the cake being effective for use as animal feed.

(111) The method of aspect 110, further comprising drying the cake, the dried cake effective as a dry animal feed.

(112) The method of aspect 110 or 111, wherein the animal feed contains protein, fat, carbohydrate, and/or minerals, e.g., from about 30 wt. % to about 90 wt. % protein, from about 1 wt. % to about 12 wt. % fat, from about 5 wt. % to about 60 wt. % carbohydrate (e.g., from about 15 wt. % to about 60 wt. %, or from about 5 wt. % to about 15 wt. %), and/or from about 1 wt. % to about 20 wt. % minerals such as sodium, potassium, copper etc., such as about 86% protein, about 2% fat, about 2% minerals, and/or about 10% carbohydrate.

(113) The method of any one of aspect 110-112, wherein the syngas contains from about 5 vol. % to about 80 vol. % of H₂, or from about 50 vol. % to about 80 vol. % of H₂.

(114) The method of any one of aspect 110-113, wherein the syngas contains from about 3 vol. % to about 85 vol. % of CO, e.g., from about 10 vol. % to about 50 vol. % of CO.

(115) The method of any one of aspects 110-114, wherein the syngas contains from about 0 vol. % to about 45 vol. % of CO₂, e.g., from about 3 vol. % to about 25 vol. % of CO₂.

(116) The method of any one of aspects 110-114, wherein the syngas contains from about 3 vol. % to about 45 vol. % of CO₂, e.g., from about 3 vol. % to about 25 vol. % of CO₂.

(117) The method of any one of aspects 110-116, wherein the H₂-enriched syngas contains at least about 50 vol. % of H₂, e.g., from about 50 vol. % to about 85 vol. %, or from about 60 vol. % to about 70 vol. % of H₂.

(118) The method of any one of aspects 110-117, wherein the H₂-enriched syngas contains from about 3 vol. % to about 50 vol. % of CO, e.g., from about 25 vol. % to about 35 vol. % of CO.

(119) The method of any one of aspects 110-118, wherein the H₂-enriched syngas contains from about 0 vol. % to about 15 vol. % of CO₂, e.g., from about 3 vol. % to about 5 vol. % of CO₂.

(120) The method of any one of aspects 110-118, wherein the H₂-enriched syngas contains from about 3 vol. % to about 15 vol. % of CO₂, e.g., from about 3 vol. % to about 5 vol. % of CO₂.

(121) The method of any one of aspects 110-120, wherein the syngas has an e/C of at least about 2, e.g., from about 2 to about 6.

(122) The method of any one of aspects 110-121, wherein the H₂-enriched syngas has an e/C of about 6 or less, e.g., from about 5.7 to about 6.

(123) The method of any one of aspects 110-122, wherein the oxygenated product is ethanol.

(124) The method of any one of aspects 110-123, wherein the oxygenated product is acetic acid, butyrate, butanol, propionate, propanol, or any combination thereof.

(125) The method of any one of aspects 110-124, the method further comprising separating the oxygenated product from the broth.

(126) The method of aspect 125, wherein the oxygenated product is separated by fractional distillation, evaporation, pervaporation, gas stripping, phase separation, and extractive fermentation, including for example, liquid-liquid extraction, or any combination thereof.

(127) The method of any one of aspects 110-126, wherein the bacteria comprises Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

(128) The method of any one of aspects 110-127, wherein the enriching comprises mixing the syngas with H₂-rich tail gas.

(129) The method of aspect 128, wherein the H₂-rich tail gas contains at least about 50 vol. % of H₂, e.g., from about 50 vol. % to about 85 vol. %, or from about 60 vol. % to about 70 vol. % of H₂.

(130) The method of aspects 128 or 129, wherein the H₂-rich tail gas is derived from purge gas from a coal derived chemical production process, such as purge gas from coal to methanol production, purge gas from coal to synthetic ammonia production, purge gas from coal to acetic acid production, purge gas from coal to ethylene glycol production, purge gas from coal to synthetic natural gas production, purge gas from coal to liquid production, coke oven gas, or any combination thereof.

(131) The method of any one of aspects 110-129, wherein the syngas contains at least about 15 vol. % of CO₂, and the enriching comprises adding H₂-rich industrial tail gas and steam to the syngas to effect a reverse water gas shift to increase the e/C to a value of from about 5.7 to about 6.

(132) The method of any one of aspects 110-129, wherein the syngas contains at least about 0 vol. % of CO₂, and the enriching comprises adding H₂-rich industrial tail gas and steam to the syngas to effect a reverse water gas shift to increase the e/C to a value of from about 5.7 to about 6.

(133) The method of any one of aspects 110-132, wherein the syngas contains at least about 15 vol. % of CO₂, and the enriching comprises adding H₂-rich industrial tail gas to the syngas to effect a reverse water gas shift to increase the amount of the H₂ to at least about 50 vol. %, e.g., from about 50 vol. % to about 70 vol. %, or from about 60 vol. % to about 70 vol. % of H₂.

(134) The method of any one of aspects 110-129, wherein the syngas contains at least about 35 vol. % of CO, and the enriching comprises adding steam to the syngas to effect a water gas shift to increase the e/C to a value of from about 5.7 to about 6.

(135) The method of any one of aspects 110-129, wherein the syngas contains at least about 35 vol. % of CO, and the enriching comprises adding steam to the syngas to effect a water gas shift to increase the amount of the H₂ to at least about 50 vol. %, e.g., from about 50 vol. % to about 70 vol. %, or from about 60 vol. % to about 70 vol. % of H₂.

(136) The method of any one of aspects 110-129, wherein the syngas contains at least about 35 vol. % of CO, and the enriching comprises adding H₂ from a renewable source to the syngas to increase the e/C to a value of from about 5.7 to about 8.

(137) The method of any one of aspects 110-129, wherein the syngas contains at least about 35 vol. % of CO, and the enriching comprises adding H₂ from a renewable source to the syngas to increase the e/C to a value of from about 5.7 to about 6.

(138) The method of any one of aspects 110-129, wherein the syngas contains at least about 35 vol. % of CO, and the enriching comprises adding H₂ from a renewable source to the syngas to increase the amount of the H₂ to at least about 50 vol. %, e.g., from about 50 vol. % to about 70 vol. %, or from about 60 vol. % to about 70 vol. % of H₂.

(139) The method of any one of aspects 110-138, wherein the syngas is coal derived syngas.

(140) The method of aspects 138 or 139, wherein the renewable source for the H₂ is solar, wind, or a combination thereof.

(141) A method of preparing fertilizer, the method comprising: (a) providing a syngas comprising at least two of the following components: CO, CO₂, and H₂; (b) enriching the H₂ content in the syngas to form a H₂-enriched syngas, e.g., (i) to at least about 50 vol. % of H₂, such as from about 50 vol. % to about 85 vol. %, from about 50 vol. % to about 70 vol. %, or from about 60 vol. % to about 70 vol. % of H₂, and/or (ii) to an e/C of at least about 5.7, such as from about 5.7 to about 8; (c) fermenting the H₂-enriched syngas with bacteria, such as acetogenic carboxydotrophic bacteria, in a liquid medium to form a broth in a bioreactor to produce an oxygenated product and a solid byproduct in the broth; (d) removing the oxygenated product from the broth to produce an oxygenated product-depleted broth; and (e) removing the solid byproduct from the broth and/or the oxygenated product-depleted broth to produce a cake and a clarified stream filtrate, the cake being effective for use as a fertilizer.

(142) A method of preparing fertilizer, the method comprising: (a) providing a syngas comprising at least two of the following components: CO, CO₂, and H₂; (b) enriching the H₂ content in the syngas to form a H₂-enriched syngas, e.g., (i) to at least about 50 vol. % of H₂, such as from about 50 vol. % to about 85 vol. %, from about 50 vol. % to about 70 vol. %, or from about 60 vol. % to about 70 vol. % of H₂, and/or (ii) to an e/C of at least about 5.7, such as from about 5.7 to about 6; (c) fermenting the H₂-enriched syngas with bacteria, such as acetogenic carboxydotrophic bacteria, in a liquid medium to form a broth in a bioreactor to produce an oxygenated product and a solid byproduct in the broth; (d) removing the oxygenated product from the broth to produce an oxygenated product-depleted broth; and (e) removing the solid byproduct from the broth and/or the oxygenated product-depleted broth to produce a cake and a clarified stream filtrate, the cake being effective for use as a fertilizer.

(143) The method of aspect 141, further comprising drying the cake, the dried cake effective as a dry fertilizer.

(144) The method of aspect 141 or 143, wherein the fertilizer contains protein, fat, carbohydrate, and/or minerals, e.g., from about 30 wt. % to about 90 wt. % protein, from about 1 wt. % to about 12 wt. % fat, from about 5 wt. % to about 60 wt. % carbohydrate (e.g., from about 15 wt. % to about 60 wt. %, or from about 5 wt. % to about 15 wt. %), and/or from about 1 wt. % to about 20 wt. % minerals such as sodium, potassium, copper etc., such as about 86% protein, about 2% fat, about 2% minerals, and/or about 10% carbohydrate.

(145) The method of any one of aspect 141-144, wherein the syngas contains from about 5 vol. % to about 80 vol. % of H₂, or from about 50 vol. % to about 80 vol. % of H₂.

(146) The method of any one of aspect 141-145, wherein the syngas contains from about 0 vol. % to about 85 vol. % of CO, e.g., from about 10 vol. % to about 50 vol. % of CO.

(147) The method of any one of aspect 141-145, wherein the syngas contains from about 3 vol. % to about 85 vol. % of CO, e.g., from about 10 vol. % to about 50 vol. % of CO.

(148) The method of any one of aspects 141-147, wherein the syngas contains from about 0 vol. % to about 45 vol. % of CO₂, e.g., from about 3 vol. % to about 25 vol. % of CO₂.

(149) The method of any one of aspects 141-148, wherein the H₂-enriched syngas contains at least about 50 vol. % of H₂, e.g., from about 50 vol. % to about 85 vol. %, or from about 60 vol. % to about 70 vol. % of H₂.

(150) The method of any one of aspects 141-149, wherein the H₂-enriched syngas contains from about 3 vol. % to about 50 vol. % of CO, e.g., from about 25 vol. % to about 35 vol. % of CO.

(151) The method of any one of aspects 141-150, wherein the H₂-enriched syngas contains from about 0 vol. % to about 15 vol. % of CO₂, e.g., from about 3 vol. % to about 5 vol. % of CO₂.

(152) The method of any one of aspects 141-151, wherein the syngas has an e/C of at least about 2, e.g., from about 2 to about 8.

(153) The method of any one of aspects 141-151, wherein the syngas has an e/C of at least about 2, e.g., from about 2 to about 6.

(154) The method of any one of aspects 141-153, wherein the H₂-enriched syngas has an e/C of about 6 or less, e.g., from about 5.7 to about 6.

(155) The method of any one of aspects 141-154, wherein the oxygenated product is ethanol.

(156) The method of any one of aspects 141-155, wherein the oxygenated product is acetic acid, butyrate, butanol, propionate, propanol, or any combination thereof.

(157) The method of any one of aspects 141-156, the method further comprising separating the oxygenated product from the broth.

(158) The method of aspect 157, wherein the oxygenated product is separated by fractional distillation, evaporation, pervaporation, gas stripping, phase separation, and extractive fermentation, including for example, liquid-liquid extraction, or any combination thereof.

(159) The method of any one of aspects 141-158, wherein the bacteria comprises Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

(160) The method of any one of aspects 141-159, wherein the enriching comprises mixing the syngas with H₂-rich tail gas.

(161) The method of aspect 160, wherein the H₂-rich tail gas contains at least about 50 vol. % of H₂, e.g., from about 50 vol. % to about 85 vol. %, or from about 60 vol. % to about 70 vol. % of H₂.

(162) The method of aspects 160 or 161, wherein the H₂-rich tail gas is derived from purge gas from a coal derived chemical production process, such as purge gas from coal to methanol production, purge gas from coal to synthetic ammonia production, purge gas from coal to acetic acid production, purge gas from coal to ethylene glycol production, purge gas from coal to synthetic natural gas production, purge gas from coal to liquid production, coke oven gas, or any combination thereof.

(163) The method of any one of aspects 141-161, wherein the syngas contains at least about 15 vol. % of CO₂, and the enriching comprises adding H₂-rich industrial tail gas and steam to the syngas to effect a reverse water gas shift reaction to convert CO₂ to CO and optionally excess H₂ added to increase the e/C to a value of from about 5.7 to about 8.

(164) The method of any one of aspects 141-161, wherein the syngas contains at least about 15 vol. % of CO₂, and the enriching comprises adding H₂-rich industrial tail gas and steam to the syngas to effect a reverse water gas shift reaction to convert CO₂ to CO and optionally excess H₂ added to increase the e/C to a value of from about 5.7 to about 6.

(165) The method of any one of aspects 141-161, wherein the syngas contains at least about 0 vol. % of CO₂, and the enriching comprises adding H₂-rich industrial tail gas and steam to the syngas to effect a reverse water gas shift reaction to convert CO₂ to CO and optionally excess H₂ added to increase the e/C to a value of from about 5.7 to about 8.

(166) The method of any one of aspects 141-161, wherein the syngas contains at least about 0 vol. % of CO₂, and the enriching comprises adding H₂-rich industrial tail gas and steam to the syngas to effect a reverse water gas shift reaction to convert CO₂ to CO and optionally excess H₂ added to increase the e/C to a value of from about 5.7 to about 6.

(167) The method of any one of aspects 141-161, wherein the syngas contains at least about 15 vol. % of CO₂, and the enriching comprises adding H₂-rich industrial tail gas to the syngas to effect a reverse water gas shift reaction to convert CO₂ to CO and optionally excess H₂ added to increase the amount of the H₂ to at least about 50 vol. %, e.g., from about 50 vol. % to about 70 vol. %, or from about 60 vol. % to about 70 vol. % of H₂.

(168) The method of any one of aspects 141-161, wherein the syngas contains at least about 35 vol. % of CO, and the enriching comprises adding steam to the syngas to effect a water gas shift to increase the e/C to a value of from about 5.7 to about 8.

(169) The method of any one of aspects 141-161, wherein the syngas contains at least about 35 vol. % of CO, and the enriching comprises adding steam to the syngas to effect a water gas shift to increase the e/C to a value of from about 5.7 to about 6.

(170) The method of any one of aspects 141-161, wherein the syngas contains at least about 35 vol. % of CO, and the enriching comprises adding steam to the syngas to effect a water gas shift to increase the amount of the H₂ to at least about 50 vol. %, e.g., from about 50 vol. % to about 70 vol. %, or from about 60 vol. % to about 70 vol. % of H₂.

(171) The method of any one of aspects 141-161, wherein the syngas contains at least about 35 vol. % of CO, and the enriching comprises adding H₂ from a renewable source to the syngas to increase the e/C to a value of from about 5.7 to about 6.

(172) The method of any one of aspects 141-161, wherein the syngas contains at least about 35 vol. % of CO, and the enriching comprises adding H₂ from a renewable source to the syngas to increase the amount of the H₂ to at least about 50 vol. %, e.g., from about 50 vol. % to about 70 vol. %, or from about 60 vol. % to about 70 vol. % of H₂.

(173) The method of any one of aspects 141-161, wherein the syngas is coal derived syngas.

(174) The method of aspects 171 or 172, wherein the renewable source for the H₂ is solar, wind, or a combination thereof.

It shall be noted that the preceding aspects are illustrative and not limiting. Other exemplary combinations are apparent from the entirety of the description herein. It will also be understood by one of ordinary skill in the art that various aspects may be used in various combinations with the other aspects provided herein.

The following examples further illustrate the disclosure but, of course, should not be construed as in any way limiting its scope.

### Example 1

This example sets forth experimental and comparative experiments that demonstrate processes for the use of use of purge gases associated with synthetic methanol production to enrich hydrogen content of syngas derived from coal.

Production of synthetic methanol is accompanied by a purge gas that contains 65-80% H₂ (as seen in, e.g., Table 1). Syngas from coal gasification (H₂:CO:CO₂:CH₄, 37:38:21:4%, respectively) is mixed with purge gas derived from synthetic methanol production to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of coal derived syngas and synthetic methanol purge gas is efficiently converted to ethanol via fermentation.

### Example 2

This example sets forth experimental and comparative experiments that demonstrate processes for the use of use of purge gases associated with synthetic methanol production to enrich hydrogen content of syngas derived from renewable sources.

Production of synthetic methanol is accompanied by a purge gas that contains 65-80% H₂ (as seen in, e.g., Table 1). Syngas from biomass or municipal waste gasification (H₂:CO:CO₂:CH₄, 37:38:21:4%, respectively) is mixed with purge gas derived from synthetic methanol production to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from renewable sources and synthetic methanol purge gas is efficiently converted to ethanol via fermentation.

### Example 3

This example sets forth experimental and comparative experiments that demonstrate processes for the use of use of purge gases associated with synthetic ammonia production to enrich hydrogen content of syngas derived from coal.

Production of synthetic ammonia is accompanied by a purge gas that contains 60-70% H₂ (as seen in, e.g., Table 2). Syngas from coal gasification (H₂:CO:CO₂:CH₄, 37:38:21:4%, respectively) is mixed with purge gas derived from synthetic ammonia production to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from coal and synthetic ammonia purge gas is efficiently converted to ethanol via fermentation.

### Example 4

This example sets forth experimental and comparative experiments that demonstrate processes for the use of use of purge gases associated with synthetic ammonia production to enrich hydrogen content of syngas derived from renewable sources. Production of synthetic ammonia is accompanied by a purge gas that contains 60-70% H₂ (as seen in, e.g., Table 2). Syngas from biomass or municipal solid waste gasification (H₂:CO:CO₂:CH₄, 37:38:21:4%, respectively) is mixed with purge gas derived from synthetic ammonia production to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from renewable sources and synthetic ammonia purge gas is efficiently converted to ethanol via fermentation.

### Example 5

This example sets forth experimental and comparative experiments that demonstrate processes for the use of use of purge gases associated with synthetic ethylene glycol production to enrich hydrogen content of syngas derived from coal.

Production of synthetic acetic ethylene glycol is accompanied by a H₂-rich purge gas that contains 70-80% H₂ (as seen in, e.g., Table 7). Syngas from coal gasification (H₂:CO:CO₂:CH₄, 37:38:21:4%, respectively) is mixed with H₂-rich purge gas derived from synthetic ethylene glycol production to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from coal and H₂-rich purge gas derived from production of ethylene glycol is efficiently converted to ethanol via fermentation.

### Example 6

This example sets forth experimental and comparative experiments that demonstrate processes for the use of use of H₂-rich purge gases associated with synthetic ethylene glycol production to enrich hydrogen content of syngas derived from renewable sources.

Production of synthetic acetic ethylene glycol is accompanied by a H₂-rich purge gas that contains 70-80% H₂ (as seen in, e.g., Table 7). Syngas from biomass or municipal solid waste gasification (H₂:CO:CO₂:CH₄, 37:38:21:4%, respectively) is mixed with H₂-rich purge gas derived from synthetic ethylene glycol production to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from renewable sources and H₂-rich purge gas derived from synthetic ethylene glycol production is efficiently converted to ethanol via fermentation.

### Example 7

This example sets forth experimental and comparative experiments that demonstrate processes for the use of coke oven gas to enrich hydrogen content of syngas derived from coal.

Coke oven gas contains 55-60% H₂ (as seen in, e.g., Table 9). Syngas from coal gasification (H₂:CO:CO₂:CH₄, 37:38:21:4%, respectively) is mixed with coke oven gas to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from coal and coke oven gas is efficiently converted to ethanol via fermentation.

### Example 8

This example sets forth experimental and comparative experiments that demonstrate processes for the use of coke oven gas to enrich hydrogen content of syngas derived from renewable sources.

Coke oven gas contains 55-60% H₂ (as seen in, e.g., Table 9). Syngas from biomass or municipal solid waste gasification (H₂:CO:CO₂:CH₄, 37:38:21:4%, respectively) is mixed with coke oven gas to generate a blended syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from a mixture of syngas derived from renewable sources and coke oven gas is efficiently converted to ethanol via fermentation.

### Example 9

This example sets forth experimental and comparative experiments that demonstrate processes for the use of use of CO₂-rich purge gases and high H₂ purge gas to produce a syngas suitable for efficient ethanol production.

Gasification of coal is accompanied by an “acid gas” purge gas that contains 95-99% CO₂% (as seen in, e.g., Table 3). Production of synthetic methanol is accompanied by a purge gas that contains 65-80% H₂ (as seen in, e.g., Table 1). This CO₂-rich purge gas is then blended with the H₂-rich purge stream, and subjected to a reverse water gas shift to produce a CO-enriched gas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended syngas derived from CO₂-rich acid gas and the purge gas from synthetic methanol production is efficiently converted to ethanol via fermentation.

### Example 10

This example sets forth experimental and comparative experiments that demonstrate processes for the use of use of CO₂-rich purge gases and high H₂ purge gas to produce a syngas suitable for efficient ethanol production.

Gasification of coal is accompanied by an “acid gas” purge gas that contains 95-99%% CO₂% (as seen in, e.g., Table 3). Production of synthetic ammonia is accompanied by a purge gas that contains 60-70% H₂ (as seen in, e.g., Table 2). The CO₂-rich acid gas is then blended with the H₂-rich purge stream, and subjected to a reverse water gas shift to produce a CO-enriched gas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended reverse water-gas shifted syngas derived from CO₂-rich acid gas and the purge gas from synthetic ammonia production is efficiently converted to ethanol via fermentation.

### Example 11

This example sets forth experimental and comparative experiments that demonstrate processes for the use of use of CO₂-rich purge gases and high H₂ purge gas to produce a syngas suitable for efficient ethanol production.

Gasification of coal is accompanied by an “acid gas” purge gas that contains 98.8% CO₂% (as seen in, e.g., Table 3). Coke oven gas contains 55-60% H₂ (as seen in, e.g., Table 9). The CO₂-rich acid gas is then blended with the H₂-rich coke oven gas, and subjected to a reverse water gas shift to produce a CO-enriched gas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then recovered from the removed broth via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that blended reverse water-gas shifted syngas derived from CO₂-rich acid gas and coke oven gas is efficiently converted to ethanol via fermentation.

### Example 12

This example sets forth experimental and comparative experiments that demonstrate processes for the use of use of CO-rich calcium carbide furnace tail gas for ethanol production.

Calcium carbide furnace purge gas contains 75-85% CO (as seen in, e.g., Table 8). This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that calcium carbide furnace tail gas is efficiently converted to ethanol via fermentation.

### Example 13

This example sets forth experimental and comparative experiments that demonstrate processes for the use of reverse water gas-shifted CO-rich calcium carbide furnace tail gas for ethanol production.

Calcium carbide furnace purge gas contains 75-85% CO (as seen in, e.g., Table 8). This syngas is mixed with steam and subjected to water gas shift to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that calcium carbide furnace tail gas is converted to a syngas via water gas shift that is efficiently converted to ethanol.

### Example 14

This example sets forth experimental and comparative experiments that demonstrate processes for the use of CO-rich calcium carbide furnace tail gas and renewable H₂ for ethanol production.

Calcium carbide furnace purge gas contains 75-85% CO (as seen in, e.g., Table 8). This gas is blended with renewable H₂ derived from electrolysis using green energy to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that a syngas derived from mixing calcium carbide furnace tail gas and renewable H₂ is efficiently converted to ethanol.

### Example 15

This example sets forth experimental and comparative experiments that demonstrate processes for the use of use of CO-rich purge gas derived from synthetic acetic acid production for ethanol production.

High pressure purge gas associated with production of synthetic acetic acid contains 70-80% CO (as seen in, e.g., Table 4). This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that purge gas derived from synthetic acetic acid production is efficiently converted to ethanol via fermentation.

### Example 16

This example sets forth experimental and comparative experiments that demonstrate processes for the use of reverse water gas-shifted CO-rich purge gas derived from synthesis of acetic acid for ethanol production.

High pressure purge gas derived from synthetic production if acetic acid contains 70-80% CO (as seen in, e.g., Table 4). This syngas is mixed with steam and subjected to water gas shift to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that synthetic acetic acid purge gas is converted to a syngas via water gas shift that is efficiently converted to ethanol.

### Example 17

This example sets forth experimental and comparative experiments that demonstrate processes for the use of purge gas derived from synthetic acetic production and renewable H₂ for ethanol production.

Calcium carbide furnace purge gas contains 70-80% CO (as seen in, e.g., Table 8). This gas is blended with renewable H₂ derived from electrolysis using green energy to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that a syngas derived from mixing calcium purge gas from acetic acid synthesis and renewable H₂ is efficiently converted to ethanol.

### Example 18

This example sets forth experimental and comparative experiments that demonstrate processes for the use of use of CO-rich purge gas derived from synthetic ethylene glycol production for ethanol production.

Purge gas associated with production of synthetic ethylene glycol contains 65-75% CO (as seen in, e.g., Table 6). This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that purge gas derived from synthetic ethylene glycol production is efficiently converted to ethanol via fermentation.

### Example 19

This example sets forth experimental and comparative experiments that demonstrate processes for the use of reverse water gas-shifted CO-rich purge gas derived from synthesis of acetic acid for ethanol production.

Purge gas derived from synthetic production of ethylene glycol contains 65-75% CO (as seen in, e.g., Table 6). This syngas is mixed with steam and subjected to water gas shift to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that synthetic ethylene glycol purge gas is converted to a syngas via water gas shift that is efficiently converted to ethanol.

### Example 20

This example sets forth experimental and comparative experiments that demonstrate processes for the use of purge gas derived from synthetic ethylene glycol production and renewable H₂ for ethanol production.

Purge gas from ethylene glycol production contains 65-75% CO (as seen in, e.g., Table 6). This gas is blended with renewable H₂ derived from electrolysis using green energy to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that a syngas derived from mixing purge gas from ethylene glycol synthesis and renewable H₂ is efficiently converted to ethanol.

### Example 21

This example sets forth experimental and comparative experiments that demonstrate processes for the use of renewable H₂ to enrich hydrogen content of syngas derived from coal.

Syngas from coal gasification (H₂:CO:CO₂:CH₄, 37:38:21:4%, respectively) is mixed with renewable H₂ derived from electrolysis using green energy to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that a syngas derived from mixing coal-derived syngas and renewable H₂ is efficiently converted to ethanol.

### Example 22

This example sets forth experimental and comparative experiments that demonstrate processes for the use of renewable H₂ to enrich hydrogen content of syngas derived from renewable sources.

Syngas from biomass or municipal waste gasification (H₂:CO:CO₂:CH₄, 37:38:21:4%, respectively) is mixed with renewable H₂ derived from electrolysis using green energy to produce a syngas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that renewable H₂ is used to enrich the hydrogen content of syngas derived from renewable sources, and that this syngas is efficiently converted to ethanol.

### Example 23

This example sets forth experimental and comparative experiments that demonstrate processes for the use of use of CO₂-rich purge gases derived from coal gasification and renewable H₂ to produce a syngas suitable for efficient ethanol production.

Gasification of coal is accompanied by an “acid gas” purge gas that contains 98.8% CO₂% (as seen in, e.g., Table 3). This CO₂-rich purge gas is then blended with the H₂ derived from hydrolysis using renewable energy, and subjected to a reverse water gas shift to produce a CO-enriched gas with an e/C of 5.96. This syngas is then fed to a steady state continuous fermentation in a bioreactor containing a carboxytrophic homoacetogen operated at a pH<6 and a hydraulic retention time (HRT) of ≤3 days. Ethanol is then removed from the reactor via distillation.

The removed broth and cells are subjected to wastewater treatment, or biosolids removed and the broth returned to the reactor. The recovered biosolids are disposed of via wastewater treatment or addition to a landfill. Alternatively, the biosolids are concentrated, dried and used as animal feed, or for land application as fertilizer.

The results demonstrate that CO₂ rich purge gases associated with coal gasification and renewable H₂ can be subjected to reverse water gas shift to produce a syngas that is efficiently converted to ethanol.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing an oxygenated product, the method comprising:
a. providing a syngas comprising at least two of the following components: CO, CO₂, and H₂;
b. enriching the H₂ content in the syngas to form a hydrogen-enriched substrate gas; and
c. fermenting the H₂-enriched substrate gas with acetogenic carboxydotrophic bacteria in a liquid medium to produce a broth containing the oxygenated product.

2. The method of claim 1, wherein the hydrogen-enriched syngas has from about 50 vol. % of H₂ to about 85 vol. % of H₂.

3. The method of claim 1, wherein the H₂-enriched substrate gas contains from about 3 vol. % to about 50 vol. % of CO.

4. The method of claim 1, wherein the H₂-enriched substrate gas contains from about 0 vol. % to about 15 vol. % of CO₂.

5. The method of claim 1, wherein the oxygenated product is ethanol.

6. The method of claim 1, wherein the oxygenated product is acetic acid, butyrate, butanol, propionate, propanol, or any combination thereof.

7. The method of claim 1, the method further comprising separating the oxygenated product from the broth by fractional distillation, evaporation, pervaporation, gas stripping, phase separation, extractive fermentation, or any combination thereof.

8. The method of claim 1, wherein the bacteria are an acetogenic carboxydotroph.

9. The method of claim 1, wherein the bacteria comprise Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

10. The method of claim 1, wherein the enriching comprises mixing the syngas with H₂-rich tail gas.

11. The method of claim 1, wherein the syngas is coal derived syngas.

12. The method of claim 1, wherein the syngas contains at least about 35 vol. % of CO, and the enriching comprises adding H₂ from a renewable source to the syngas to increase the e/C to a value of from about 5.7 to about 8.0.

13. The method of claim 12, wherein the renewable source for the H₂ generates electricity to run electrolysis to produce renewable hydrogen.

14. The method of claim 1, wherein the H₂ content in the syngas is enriched without the removal of hydrogen sulfide.

15. The method of claim 1, wherein the syngas has an e/C of from about 5.7 to about 8.

16. The method of claim 15, wherein the hydrogen-enriched syngas has from about 50 vol. % of H₂ to about 85 vol. % of H₂.

17. The method of claim 15, wherein the H₂-enriched substrate gas contains from about 3 vol. % to about 50 vol. % of CO.

18. The method of claim 15, wherein the H₂-enriched substrate gas contains from about 0 vol. % to about 15 vol. % of CO₂.

19. The method of claim 15, wherein the oxygenated product is ethanol, acetic acid, butyrate, butanol, propionate, propanol, or any combination thereof.

20. The method of claim 15, wherein the syngas contains at least about 35 vol. % of CO, and the enriching comprises adding H₂ from a renewable source to the syngas, and wherein the renewable source for the H₂ generates electricity to run electrolysis to produce the hydrogen.

21. The method of claim 15, wherein the bacteria comprise Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

22. The method of claim 15, wherein the H₂ content in the syngas is enriched without the removal of hydrogen sulfide.

23. A method of preparing an oxygenated product, the method comprising:
a. providing a syngas comprising at least two of the following components: CO, CO₂, and H₂;
b. enriching the H₂ content in the syngas to form a hydrogen-enriched substrate gas having at least about 50 vol. % of H₂;
c. fermenting the H₂-enriched substrate gas with bacteria in a liquid medium to produce a broth containing the oxygenated product.

24. The method of claim 23, wherein the hydrogen-enriched syngas has from about 50 vol. % of H₂ to about 85 vol. % of H₂.

25. The method of claim 23, wherein the H₂-enriched substrate gas contains from about 3 vol. % to about 50 vol. % of CO.

26. The method of claim 23, wherein the H₂-enriched substrate gas contains from about 0 vol. % to about 15 vol. % of CO₂.

27. The method of claim 23, the method further comprising separating the oxygenated product from the broth by fractional distillation, evaporation, pervaporation, gas stripping, phase separation, extractive fermentation, or any combination thereof.

28. The method of claim 27, wherein the oxygenated product is ethanol, acetic acid, butyrate, butanol, propionate, propanol, or any combination thereof.

29. The method of claim 28, wherein the bacteria are an acetogenic carboxydotroph.

30. The method of claim 28, wherein the bacteria comprise Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

31. The method of claim 29, wherein the H₂ content in the syngas is enriched without the removal of hydrogen sulfide.

32. A method of renewably preparing an oxygenated product, the method comprising:
a. providing a syngas comprising at least two of the following compounds: CO, CO₂, and H₂;
b. adding H₂ from a renewable source to the syngas to form an H₂ enriched substrate gas;
c. fermenting the H₂-enriched substrate gas with bacteria in a liquid medium to produce a broth containing the oxygenated product.

33. The method of claim 32, wherein the renewable source for the H₂ generates electricity to run electrolysis to produce renewable hydrogen.

34. The method of claim 32, wherein the H₂ content in the syngas is enriched without the removal of hydrogen sulfide.

35. The method of claim 32, wherein the renewable source of H₂ is formed from municipal waste.

36. The method of claim 32, wherein the hydrogen-enriched syngas contains from about 50 vol. % of H₂ to about 85 vol. % of H₂.

37. The method of claim 32, wherein the syngas has an e/C of from about 5.7 to about 8.

38. The method of claim 32, wherein the oxygenated product is acetic acid, butyrate, butanol, propionate, propanol, or any combination thereof.

39. The method of claim 38, wherein the bacteria comprise Clostridium, Moorella, Pyrococcus, Eubacterium, Desulfobacterium, Carboxydothermus, Acetogenium, Acetobacterium, Acetoanaerobium, Butyribacterium, Peptostreptococcus, or any combination thereof.

40. The method of claim 39, the method further comprising separating the oxygenated product from the broth by fractional distillation, evaporation, pervaporation, gas stripping, phase separation, extractive fermentation, or any combination thereof.
